(19) United States
(12) Patent Application Publication
(10) Pub. No.: US 2016/0272620 A1
(43) Pub. Date: Sep. 22, 2016

(54) NOVEL UNCHARGED REACTIVATORS AGAINST OP-INHIBITION OF HUMAN ACETYLCHOLINESTERASE

(71) Applicants: UNIVERSITE DE STRASBOURG, Strasbourg (FR); COMMISSARIAT A L'ENERGIE ATOMIQUE ET AUX ENERGIES ALTERNATIVES, Paris (FR); CENTRE NATIONAL DE LA RECHERCHE SCIENTIFIQUE, Paris (FR); UNIVERSITE DE ROUEN, Mont Saint Aignan (FR); L'ETAT (MINISTÈRE DE LA DÉFENSE), REPRÉSENTÉ PAR L E DIRECTEUR CENTRAL DU SERVICE DE SANTÉ DES ARMÉE, Paris Cedex 12 (FR)

(72) Inventors: Rachid BAATI, SOUFFELWEYERSHEIM (FR); Maria KLIACHYNA, ILLKIRCH GRAFFENSTADEN (FR); Valentin NUSSBAUM, SAUSHEIM (FR); Pierre-Yves RENARD, PARIS (FR); Ludovic JEAN, ROUEN (FR); Martin WEIK, SAINT EGREVE (FR); Florian NACHON, GIERES (FR); Melanie TOUVREY, LE VERSOUD (FR); Melanie FRIEDEL, GRENOBLE (FR); Julien RENOU, LA FRENAYE (FR); Tristan VERDELET, ROUEN (FR); Guillaume MERCEY, MONTAURE (FR); Jacques-Philippe COLLETIER, BOURGOIN JALLIEU (FR); Benoit SANSON, New York, NY (US); Gianluca SANTONI, GRENOBLE (FR)

(21) Appl. No.: 15/037,169

(22) PCT Filed: Nov. 19, 2014
(86) PCT No.: PCT/EP2014/075046

§ 371 Date: May 17, 2016

(30) Foreign Application Priority Data
Nov. 19, 2013 (EP) 13306584.7

(51) Int. Cl. C07D 401/12

(52) U.S. Cl. CPC C07D 401/12

(57) ABSTRACT

Novel uncharged reactivators of human acetylcholinesterase, pharmaceutical compositions including the compounds, and their use for reactivating human acetylcholinesterase inhibited by at least one organophosphorus nerve agent.

### INTRODUCTION

The present invention deals with novel uncharged reactivators of human acetylcholinesterase, pharmaceutical compositions comprising said compounds, and their use for reactivating human acetylcholinesterase inhibited by at least one organophosphorus nerve agent.

### BACKGROUND OF THE INVENTION

Organophosphorus nerve agents (OPNA) are extremely toxic compounds that include chemical warfare agents (CWA) (sarin, soman, cyclosarin, tabun, methylphosphonothioate VX) and pesticides (paraoxon, parathion, tetraethyl pyrophosphate (TEPP)). Their acute toxicity results from the irreversible inhibition of acetylcholinesterase (AChE) through phosphylation of its catalytic serine. Accumulation of neurotransmitter acetylcholine (ACh) at cholinergic synapses ensues, leading to nervous and respiratory failures. Depending on the class of OPNA and on the administrated dose, death can occur within minutes.

Due to the similarity between the chemical precursors of CWA and pesticides, and to the relatively simple chemistry involved in their synthesis, efforts to control the proliferation of these agents have proved of limited success. Illustrative examples include the terrorist attack in the Tokyo subway in 1995, the bombing of Kurd civilians during the Iraq-Iran war in 1988, and that of civilians in Syria, as reported in August 2013. Additionally, despite the international efforts aimed at regulating and lessening the use of these environmentally toxic compounds, ca. 100 different OPNA are still used intensively as pest control agents, with only anecdotal monitoring. This results in about 3,000,000 acute intoxications per year, 200,000 of which lead to death. Therefore, the development of effective measures to counteract organophosphorus (OP) poisoning remains a challenging issue to protect and treat both civilian and military populations.

The current treatment against OP poisoning consists in the administration of a combination of atropine (antimuscarinic agent) and diazepam (anticonvulsant drug), to limit convulsions, and of a standard pyridinium oxime (pralidoxime, trimedoxime, HI-6, obidoxime, or HLö-7) to reactivate AChE. Oximes exert their action on OP-inhibited AChE by attacking the phosphorus atom of the phosphylated serine, yielding to the removal of the phosphonate and recovery of the enzyme's catalytic activity. To this end, pyridinium oximes must display high nucleophilicity, which is generally attained by the formation of an oximate anion at physiological pH. As of today, however, not a single oxime has proven equally effective against all types of OP-inhibited AChE, and most are ineffective against tabun-inhibited hAChE.

Another weakness of currently approved pyridinium aldoximes is their difficulty in crossing the blood-brain barrier (BBB) owing to the permanent positive charge carried by the pyridinium nitrogen atom. For example, it was estimated using in vivo rat brain microdialysis coupled with HPLC/UV, that the BBB penetration of the most commonly used oxime, 2-PAM, is only 10%. Therefore, oximes reactivating AChE in the peripheral nervous system are not effective in the brain and, consequently, do not protect against the neurological effects of OP-exposure.

Neutral oxime-based compounds, both able to cross the BBB and to reactivate OP-inhibited AChE in the Central Nervous System, have also been synthesized.

Coupling of an oxime-based structure to a potential ligand of the peripheral site of the enzyme (PSL) was also investigated to increase affinity of the reactivator for AChE.

AChE reactivators obtained by these strategies are often less efficient than currently used pyridinium oximes. Further, these compounds are often efficient for only some types of OP-inhibited-AChE. Finally, these compounds sometimes present low affinity for the enzyme, and consequently high concentrations must be used for obtaining the desired reactivation.

In particular, tabun-hAChE is known to be reluctant to reactivation due to weak eletrophilicity and steric hindrance of the tabun-hAChE adduct. None of the previously described reactivating compounds obtained by any of the above mentioned strategies is able to reactivate all types of inhibited hAChE, in particular tabun-hAChE.

The choice of the peripheral site ligands that can be coupled to the oxime moiety to create bifunctional reactivators with enhanced affinity to hAChE as disclosed above is based on their character of inhibitor of native hAChE.

In this context, the Applicant surprisingly found that bifunctional compounds comprising a peripheral site ligand moiety with a tacrine (1,2,3,4-tetrahydroacridine) structure, a specific linker, and an oxime moiety are potent reactivators of human AChE inhibited with any type of organophosphorus compounds. Tacrines are however known to be not among the best inhibitors of hAChE.

### SUMMARY OF THE INVENTION

A first object of the present invention is a compound of formula (I)

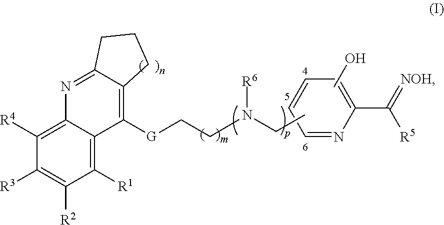

wherein
the different groups are as defined in the detailed description below.

Another object of the present invention is a pharmaceutical composition comprising at least one compound of formula (I) and at least one pharmaceutically acceptable support.

Another object of the invention is a compound or a composition according to the invention, for use as a medicament.

Another object of the invention is a compound or a composition according to the invention, for use as a reactivator of human acetylcholinesterase in vitro and/or in vivo, wherein the human acetylcholinesterase was inhibited by at least one organophosphorus nerve agent.

A last object of the invention is a compound or a composition according to the invention for use in the treatment of a nervous and/or respiratory failure due to intoxication with at least one organophosphorus nerve agent.

### BRIEF DESCRIPTION OF THE DRAWINGS

FIG. 1: Docked conformation of molecule 12 at the peripheral site of VX-hAChE.

FIG. 2: Distribution of distances between the aldoxime oxygen and the VX phosphorus analyzed along the simulation trajectory for the four different linker lengths (KM 2: —CH₂—CH₂—; KM 3: —CH₂—CH₂—CH₂—, KM 4: —CH₂—CH₂—CH₂—CH₂—, 12; KM 5: —CH₂—CH₂—CH₂—CH₂—CH₂—, 11).

### DETAILED DESCRIPTION OF THE INVENTION

A first object of the invention is a compound of formula (I):

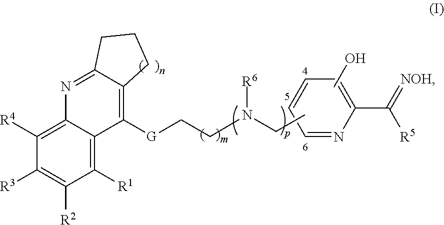

wherein
G is selected from the group consisting of CH₂, O, S, NH, NR, C(O)—NH, C(O)—NR, NH—C(O), NR—C(O), NH—C(S), NR—C(S), NH—NR, NR—NR′, NH—O, NR—O, NH—C—(O)—NH, NR—C(O)—NH, NR—C(O)—NR′, NH—C(S)—NH, NR—C(S)—NH, and NR—C(S)—NR′, preferably G is NH or S, more preferably G is NH,
wherein each R or R′ is independently selected from the group consisting of a hydrogen atom, an alkyl group, an acyl group, an aryl group and a heteroaryl group;
   n is 1, 2, 3 or 4, preferably n is 2 or 3, more preferably n is 2,
   m is 0, 1, 2, 3, or 4, preferably m is 2 or 3, more preferably m is 3,
   p is 0 or 1, preferably p is 0,
   R¹, R², R³ and R⁴ are independently selected from the group consisting of a hydrogen atom, a halogen atom, an alkyl group, an alkoxy group, an amine NHR″ group, an amide NR″—C(O)—R′″ group, an oligopeptide or a polyethyleneglycol (PEG) chain, such as a 2-hydroxyethoxy group (OCH₂CH₂OH), wherein R″ and R′″ have the same definition as R and R′,
   R⁵ is a hydrogen atom, a trifluoromethyl (CF₃) group, or a NH₂ group, preferably R⁵ is a hydrogen atom, and
   R⁶ is a hydrogen atom, an alkyl group, an aryl group or an acyl group, preferably R⁶ is an alkyl group, in particular a methyl group.

In an embodiment, the pyridine is substituted in position 6 by the tacrine-bearing group and/or in position 3 by the OH group. Preferably, both conditions are satisfied.

In an embodiment, three among R¹, R², R³ and R⁴ are hydrogen atoms. For instance, R¹, R³ and R⁴ are hydrogen atoms and R² is a halogen atom, such as a chlorine atom. In another embodiment, R¹, R², R³ and R⁴ are all hydrogen atoms.

In the present invention, an “alkyl group” is a linear, branched or cyclic saturated hydrocarbon group comprising from 1 to 10 carbon atoms, preferably from 1 to 6 carbon atoms, in particular from 1 to 3 carbon atoms. As examples of alkyl groups may be cited the methyl, ethyl, n-propyl, isopropyl, n-butyl, isobutyl, tert-butyl, n-pentyl, isopentyl, n-hexyl, and cyclohexyl groups.

An “alkoxy group” is an alkyl group, linked to the rest of the compound via an oxygen atom (ether linkage).

An “aryl group” refers to a monocyclic or polycyclic hydrocarbon aromatic group, with at least one of the rings having a system of conjugated pi electrons, which may be optionally substituted. Examples of aryl groups that can be used are optionally substituted phenyl, naphthalene and anthracene. Preferably, the aryl group is a phenyl group, optionally substituted.

A “heteroaryl group” is an aryl group, wherein at least one carbon atom is replaced with a heteroatom such as N, O, P or S. Examples of heteroaryl groups include pyrrole, thiophene, furane, pryridine, pyrimidine, pyrazine, triazine, imidazole, thiazole, oxazole, and isoxazole.

An “acyl group” is an alkyl group or an aryl group, linked to the rest of the compound via a C(═O) linkage.

A “halogen atom” is an atom of F, Cl, Br or I.

An “oligopeptide” is a polymer of 2 to 10 amino acids, linked to each other via peptidic bonds. Preferably, an oligopeptide according to the invention comprises from 2 to 6 amino acids. Examples of amino acids include alanine, arginine, asparagine, aspartate, cysteine, glutamate, glutamine, glycine, histidine, isoleucine, leucine, lysine, methionine, phenylalanine, proline, pyrrolysine, selenocysteine, serine, threonine, tryptophane, tyrosine and valine. Amino acids are preferably L-amino acids.

A “polyethyleneglycol chain” is a group of the formula [O—(CH₂)₂]ₐ—OH, wherein a is an integer from 1 to 150, preferably from 1 to 10.

An “organophosphorus nerve agent” (OPNA) is any organophosphorus compound having an effect on the nervous system. This term designates for instance chemical warfare agents, such as sarin, soman, cyclosarin, tabun, methylphosphonothioate, VX (O-ethyl S-[2-(diisopropylamino)ethyl]methylphosphonothioate) and pesticides such as paraoxon, parathion and tetraethyl pyrophosphate.

The compounds of the invention are preferably selected from:
N′,3-dihydroxy-4-{5-[(1,2,3,4-tetrahydroacridin-9-yl)amino]pentyl}pyridine-2-carboximidamide (6)
N′,3-dihydroxy-6-{5-[(1,2,3,4-tetrahydroacridin-9-yl)amino]pentyl}pyridine-2-carboximidamide (7)
(Z)—N′,3-dihydroxy-4-{[methyl({2-[(1,2,3,4-tetrahydroacridin-9-yl)amino]ethyl}) amino]methyl}pyridine-2-carboximidamide (8)
(Z)—N′,3-dihydroxy-4-{[methyl({3-[(1,2,3,4-tetrahydroacridin-9-yl)amino]propyl}) amino]methyl}pyridine-2-carboximidamide (9),
2-[(hydroxyimino)methyl]-4-{5-[(1,2,3,4-tetrahydroacridin-9-yl)amino]pentyl}pyridin-3-ol (10),
2-[(1E)-(hydroxyimino)methyl]-6-{5-[(1,2,3,4-tetrahydroacridin-9-yl)amino]pentyl}pyridin-3-ol (11)
2-[1-(hydroxyimino)methyl]-6-{4-[(1,2,3,4-tetrahydroacridin-9-yl)amino]butyl}pyridin-3-ol (12),
2-[1-(hydroxyimino)methyl]-5-{5-[(1,2,3,4-tetrahydroacridin-9-yl)amino]pentyl}pyridin-3-ol (13),
2-[1-(hydroxyimino)methyl]-6-[5-(1,2,3,4-tetrahydroacridin-9-ylsulfanyl)pentyl]pyridin-3-ol (14),
6-(4-((7-Chloro-1,2,3,4-tetrahydroacridin-9-yl)amino)butyl)-3-hydroxy-picolin-aldehyde oxime (15),
3-Hydroxy-6-(3-((1,2,3,4-tetrahydroacridin-9-yl)amino)propyl) picolinaldehyde oxime (16),
3-Hydroxy-6-(3-((7,8,9,10-tetrahydro-6H-cyclohepta[b]quinolin-11-yl)amino) propyl)picolinaldehyde oxime (17), and
3-Hydroxy-6-(4-((7,8,9,10-tetrahydro-6H-cyclohepta[b]quinolin-11-yl)amino) butyl)picolinaldehyde oxime (18).

In an embodiment, the compounds of the invention are selected among compounds 6, 7, 8, 9, 10, 11, 12 and 13. In a preferred embodiment, the compounds of the invention are selected among compounds 11 and 12. In a highly preferred embodiment, the compound of the invention is compound 12.

### Synthesis of the Compounds of the Invention

A compound of formula (I) according to the invention may be synthesized by any appropriate method known by anyone of ordinary skill in the art.

In particular, the process for the synthesis of compounds of formula (I) may comprise a step of Sonogashira coupling reaction between a halogenated pyridine derivative and a compound comprising a terminal alkyne and a tacrine moiety. The resulting alkyne may be reduced by reaction with hydrogen, for instance in presence of Pearlmann's catalyst Pd(OH)₂/C.

For the compounds of formula (I) with R⁵=H, the oxime may be formed from the corresponding aldehyde by reaction with NH₂OH, preferably as a hydrochloride, preferably in presence of a base, such as CH₃COONa.

For the compounds of formula (I) with R⁵=NH₂, the amidoxime may be formed from the corresponding nitrile by reaction with NH₂OH, preferably as a hydrochloride, preferably in presence of a base, such as pyridine.

### Pharmaceutical Use of the Compounds of the Invention

Another object of the invention is a pharmaceutical composition comprising at least one compound according to the invention and a pharmaceutically acceptable support.

The pharmaceutical composition comprising the compound of formula (I) according to the invention can be prepared by mixing the compound with a physiologically acceptable support, an excipient, a binder and/or a diluent.

The amount of compound of formula (I) in the composition according to the invention may evolve in a large scope depending upon the patient, the mode of administration and the expected effect.

The compound or composition according to the invention can be administered orally or non-orally, for instance via parenteral, intramuscular, intravenous, cutaneous, nasal or rectal route.

The pharmaceutical composition of the invention can present different forms including granules, powders, tablets, gel capsules, syrups, emulsions, suspensions, and forms used for non-oral administration, for instance injections, sprays or suppositories. These pharmaceutical forms can be prepared via known conventional techniques.

The preparation of an orally administered solid pharmaceutical form can be for instance performed by the following process: an excipient (for example lactose, sucrose, starch or mannitol), a disintegrant (for example calcium carbonate, calcium carboxymethylcellulose, alginic acid, sodium carboxymethylcellulose, colloidal silicon dioxide, sodium croscarmellose, Crospovidone, guar gum, magnesium aluminium silicate, microcrystalline cellulose, cellulose powder, pregelatinised starch, sodium alginate or starch glycolate), a binder (for example alpha-starch, gum arabic, carboxymethylcellulose, polyvinylpyrrolidone, hydroxypropylcellulose, alginic acid, carbomer, dextrin, ethylcellulose, sodium alginate, maltodextrin, liquid glucose, magnesium aluminium silicate, hydroxyethylcellulose, methylcellulose or guar gum) and a lubricant (for example talc, magnesium stearate or polyethylene 6000) are added to the active principle and the mixture obtained is then tabletted. If necessary, the tablet can be coated via the known techniques, in order to mask the taste (for example with cocoa powder, mint, borneol or cinnamon powder) or to allow enteric dissolution or sustained release of the active principles. Coating products that can be used are, for example, ethylcellulose, hydroxymethylcellulose, polyoxyethylene glycol, cellulose acetophthalate, hydroxypropylmethylcellulose phthalate and Eudragit® (methacrylic acid-acrylic acid copolymer), Opadry® (hydroxypropylmethylcellulose+macrogol+titanium oxide+lactose monohydrate). Pharmaceutically acceptable colorants may be added (for example yellow iron oxide, red iron oxide or quinoline yellow lake).

Liquid pharmaceutical forms for oral administration include solutions, suspensions and emulsions. The aqueous solutions can be obtained by dissolving the active principle in water, followed by addition of flavourings, colorants, stabilisers and/or thickeners, if necessary. In order to improve the solubility, it is possible to add ethanol, propylene glycol or any other pharmaceutically acceptable non-aqueous solvent. The aqueous suspensions for oral use can be obtained by dispersing the finely divided active principle in water with a viscous product, such as a natural or synthetic gum or resin, methylcellulose or sodium carboxymethylcellulose.

The pharmaceutical forms for injection can be obtained, for example, by the following process. The active principle is dissolved, suspended or emulsified either in an aqueous medium (for example distilled water, physiological saline or Ringer's solution) or in an oily medium (for example olive oil, sesame seed oil, cottonseed oil, corn oil or propylene glycol), with a dispersant (for example Tween® 80, HCO® 60 (Nikko Chemicals), polyethylene glycol, carboxymethylcellulose or sodium alginate), a preserving agent (for example methyl p-hydroxybenzoate, propyl p-hydroxybenzoate, benzyl alcohol, chlorobutanol or phenol), an isotonicity agent (for example sodium chloride, glycerol, sorbitol or glucose) and optionally other additives, such as, if desired, a solubilising agent (for example sodium salicylate or sodium acetate) or a stabiliser (for example human serum albumin).

Pharmaceutical forms for external use can be obtained from a solid, semi-solid or liquid composition containing the active principle. For example, to obtain a solid form, the active principle can be treated with excipients (for example lactose, mannitol, starch, microcrystalline cellulose or sucrose) and a thickener (for example natural gums, cellulose derivatives or acrylic polymers) so as to convert them into powder. The liquid pharmaceutical compositions are prepared in substantially the same way as the forms for injection, as indicated previously. The semi-solid pharmaceutical forms are preferably in the form of aqueous or oily gels or in the form of pomades. These compositions may optionally contain a pH regulator (for example carbonic acid, phosphoric acid, citric acid, hydrochloric acid or sodium hydroxide) and a preserving agent (for example a p-hydroxybenzoic acid ester, chlorobutanol or benzalkonium chloride).

Another object of the invention is a compound according to the invention, for use as a medicament.

Another object of the invention is a compound according to the invention, for in vivo and/or in vitro use as a reactivator of human acetylcholinesterase, wherein the human acetylcholinesterase was inhibited by at least one organophosphorus nerve agent.

Another object of the present invention is a compound according to the invention, for use in the treatment of a disorder caused by at least one organophosphorus nerve agent.

Another object of the present invention is a compound according to the invention, for use in the treatment of a nervous and/or respiratory failure due to intoxication with at least one organophosphorus nerve agent.

Another object of the present invention is a method for the treatment of a nervous and/or respiratory failure due to intoxication with at least one organophosphorus nerve agent, comprising administering at least one compound according to the invention.

Another object of the present invention is the use of a compound according to the invention for the preparation of a medicament for the treatment of a nervous and/or respiratory failure due to intoxication with at least one organophosphorus nerve agent.

Within the context of the invention, the term treatment denotes curative, symptomatic, and/or preventive treatments. In particular, it can refer to reducing the progression of the disease, reducing or suppressing at least one of its symptoms or complications, or improving in any way the state of health of patients.

The administration of the compounds or of the composition according to the invention may be performed either before or after the exposition of the subject to the organophosphorus nerve agent. The term “after” also includes the possibility for the administration and the exposition to the OP to be simultaneous.

In the present invention, the terms “subject” and “patient” are used indifferently and designate a human subject.

The amount of compound of formula (I) to be administered according to the invention may evolve in a large scope depending upon the patient, the mode of administration and the expected effect. In particular, the amount of compound of formula (I) may be comprised between 200 mg and 4000 mg, with up to 3 takes per day.

The compound or composition according to the invention may be co-administered with at least one other active agent, such as an antimuscarinic agent, in particular atropine, an anticonvulsant, in particular diazepam or one of its prodrugs, such as avizafone, and/or a bioscavenger able to capture and/or degrade OPs in blood, such as human butyrylcholinesterase.

The term co-administered means that the administration of the compound or composition according to the invention and that of the other active agent can be simultaneous, sequential and/or separate.

The following examples are provided as illustrative, and not limitative, of the present invention.

### EXAMPLES

### Example 1

### Synthesis of Compounds of the Invention

General Procedures

General procedure 1 for the Sonogashira coupling reaction (synthesis of compounds 20, 23, 35, 36, and 37). The flask containing solution of iodo- or bromopyridine (1 equiv) in THF/Et₃N (tetrahydrofuran/triethylamine) was evacuated and filled with argon three times before the addition of catalysts Pd(PPh₃)₄ (0.1 equiv) and CuI (0.2 equiv). After degazation with Argon the mixture was stirred at the room temperature for 5 min, then the degazed solution of alkyne (1.1 equiv) was added and the reaction mixture was stirred during 20 h at the room temperature. After concentration at reduced pressure the residue was purified by the column chromatography.

General procedure 2 for N-(diethylamino)carbamate group removal and nitrile conversion to amidoxime (synthesis of compounds 6, 8, and 9). The solution of 21, 28 or 29 (1 equiv), NH₂OH.HCl (30 equiv), and pyridine (30 equiv) in 2 mL of ethanol was refluxed during 14 h. The solvent was removed in vacuum, the residue was subjected to preparative HPLC chromatography.

General procedure 3 for the one-pot alkyne reduction and O-debenzylation (synthesis of compounds 24, 38, 39, and 40). To the degazed solution of alkyne (1 equiv) in ethyl acetate or methanol Pearlman's catalyst Pd(OH)₂/C (20%, moisture 50%, 0.3 equiv) was added. After the degazation the solution was bubbled with H₂. The reaction mixture was stirred at RT (room temperature) under H₂ (1 atm) during 24 h. The solution was filtered through Celite®, the solvent was evaporated, and the product was dried in vacuum.

General procedure 4 for the three-step transformation of the methyl ester into aldehyde (synthesis of compounds 31, 41, 42, 43 and 50). The solution of methyl ester 30, 38, 39, 40 or 49 (1 equiv), imidazole (4.15 equiv), and TBDMSCl (tert-Butyldimethylsilyl chloride) (2.4 equiv) in dry DMF (dimethylformamide) was stirred at room temperature under argon during 2 h. Ethyl acetate was added, and the organic phase was washed with water 3 times, dried over Na₂SO₄ and concentrated in vacuum. After drying in vacuum, the compound obtained was subjected without purification to the following reduction of methyl ester with DIBAL-H (diisobutylaluminum hydride). To the solution of the compound obtained (1 equiv) in dry CH₂Cl₂ at −78° C. DIBAL-H (1M solution in CH₂Cl₂, 2 equiv) was added dropwise. The reaction mixture was stirred at −78° C. for 12 min, then it was quenched with MeOH, and the cooling bath was removed. When the mixture was warmed to the room temperature, the organic phase was washed with aqueous solution of NaOH (1M), dried over Na₂SO₄ and concentrated under reduced pressure. After drying in vacuum the product obtained was subjected to the following reaction with TBAF (tetra n-butylammonium fluoride). To the solution of the compound obtained (1 equiv) in dry THF at 0° C. TBAF (1 M solution in THF, 1.1 equiv) was added, and the reaction mixture was stirred at this temperature for 1 h. After removal of the solvent under reduced pressure the residue was purified by column chromatography (silica gel).

General procedure 5 for synthesis of oximes 10, 11, 12, 13, and 14. The solution of aldehyde 31, 41, 42, 43 or 50 (1 equiv), NH₂OH.HCl (1.5 equiv), and CH₃COONa (1.5 equiv) in dry ethanol was stirred during 8 h. After concentration under reduced pressure the crude product was purified by the column chromatography or preparative HPLC.

### 1.1—Synthesis of Compound 6

### 2-Cyanopyridin-3-yl N,N-diethylcarbamate (17)

To the solution of 2-cyano-3-hydroxypyridine 16 (2.00 g, 0.01665 mol, 1 equiv) in 40 mL of pyridine at 0° C. diethylcarbamoylchloride (2.2 mL, 2.37 g, 0.01748 mol, 1.05 equiv) was added. The reaction mixture was stirred at the room temperature overnight. The solvent was removed in vacuum. The resulting crude material was treated with water. Then the mixture was neutralized with HCl, and the resulting product was extracted with ethyl ether, the extract was washed with aqueous 10% Na₂CO₃ and brine, dried over Na₂SO₄. The solvent was evaporated and product was dried in vacuum. The crude product was purified by column chromatography (silica gel, ethyl acetate/c-hexane=1/2). R_{f}=0.45 (ethyl acetate/c-hexane=2/3). Yield 3.46 g, 95%. ¹H NMR (400 MHz, CDCl₃, δ): 1.21 (3H, t, J=7.2 Hz, CH₃); 1.30 (3H, t, J=7.2 Hz, CH₃); 3.38 (2H, q, J=7.2 Hz, CH₂); 3.49 (2H, q, J=7.2 Hz, CH₂); 7.49-7.52 (1H, dd, J=8.5 Hz, J=4.6 Hz, ArH); 7.82-7.84 (1H, dd, J=8.5 Hz, J=1.3 Hz, ArH); 8.47-8.49 (1H, dd, J=4.6 Hz, J=1.3 Hz, ArH). ¹³C NMR (100 MHz, CDCl₃, δ): 13.34; 14.31; 42.61; 43.02; 114.69; 127.50; 131.23; 147.17; 151.56; 151.97. HRMS (ESI): m/z: calcd for C₁₁H₁₄N₃O₂ [M+H]⁺: 220.10860. Found 220.10826.

### 2-Cyano-4-iodopyridin-3-yl N,N-diethylcarbamate (18)

To the solution of 2-cyano-pyridine-3-yl-diethylcarbamate 17 (1.00 g, 0.00456 mol, 1 equiv) in 40 mL of THF n-BuLi solution (1.6M, in hexanes, 4.28 mL, 0.006841 mol, 1.5 equiv) was added dropwise at −78° C. After 30 min of stirring at −78° C. 10 mL of 12 solution in THF (0.006841 mol, 1.5 equiv) was added. The reaction mixture was stirred for 1 h at −78° C., then 20 mL of aqueous NH₄Cl solution (10%) was added. The organic phase was separated, the aqueous phase was extracted with ethyl ether, and the combined organic phases were washed with brine. After removal of the solvents the crude product was subjected to the column chromatography (silica gel, ethyl acetate/c-hexane=1/5). R_{f}=0.47 (ethyl acetate/c-hexane=1/3). Yield 48%. ¹H NMR (400 MHz, CDCl₃, δ): 1.24 (3H, t, J=7.2 Hz, CH); 1.36 (3H, t, J=7.2 Hz, CH₃); 3.41 (2H, q, J=7.2 Hz, CH₂); 3.53 (2H, q, J=7.2 Hz, CH₂); 7.96 (1H, d, J=5 Hz. ArH); 8.12 (1H, d, J=5 Hz, ArH). ¹³C NMR (100 MHz, CDCl₃, δ): 13.42; 14.48; 42.81; 43.29; 104.54; 113.86; 129.50; 137.85; 147.65; 151.00; 152.87. HRMS (ESI): m/z: calcd for C₁₁H₁₃IN₃O₂ [M+H]⁺: 346.00524. Found 346.00478.

### 2-Cyano-4-{5-[(1,2,3,4-tetrahydroacridin-9-yl)amino]pent-1-yn-1-yl}pyridin-3-yl N,N-diethylcarbamate (20)

Compound 20 was obtained accordingly to the general procedure 1 using 18 (0.22 g, 0.000637 mol, 1 equiv), 19 (0.202 g, 0.000701 mol, 1.1 equiv), Pd(PPh₃)₄ (0.057 g, 0.0000637 mol, 0.1 equiv), CuI (0.024 g, 0.0001275 mol, 0.2 equiv), 5 mL of Et₃N and 8 mL of THF. Compound 20 was purified by column chromatography (silica gel, gradient from ethyl acetate/c-hexane/Et₃N=20/10/1 to ethyl acetate/Et₃N=20/1). R_{f}=0.14 (CHCl₃/MeOH/Et₃N=100/5/1). Yield 0.267 g, 87%. ¹H NMR (400 MHz, CDCl₃, δ): 1.14 (3H, t, J=7.2 Hz, CH₃CH₂), 1.24 (3H, t, J=7.2 Hz, CH₃CH₂), 1.87-1.94 (6H, m), 2.54 (2H, t, J=7 Hz, CH₂), 2.69 (2H, t, J=5.6 Hz, CH₂), 3.04 (2H, t, J=5.8 Hz, CH₂), 3.30 (2H, q, J=7.2 Hz, NCH₂CH₃), 3.39 (2H, q, J=7.2 Hz, NCH₂CH₃), 3.57 (2H, t, J=7 Hz, CH₂NH), 7.31 (1H, m, ArH), 7.38 (1H, d, J=4.9 Hz, ArH), 7.53 (1H, m, ArH), 7.89 (2H, d, J=8.8 Hz, ArH), 8.40 (1H, d, J=4.9 Hz, ArH), ¹³C NMR (100 MHz, CDCl₃, δ): 13.47; 14.26; 17.59; 22.83; 23.15; 25.11; 29.90; 33.86; 42.63; 43.10; 48.07; 74.10; 82.93; 101.32; 114.31; 116.79; 120.42; 122.59; 124.30; 128.67; 128.84; 129.42; 130.09; 147.06; 147.55; 150.52; 151.61; 151.74; 158.52.

### 2-Cyano-4-(5-[(1,2,3,4-tetrahydroacridin-9-yl)amino]pentyl)pyridin-3-yl N,N-diethylcarbamate (21)

To the degazed solution of 20 (0.1 g, 0.208 mmol, 1 equiv) in 2 mL of ethyl acetate Pd/C (10%, 0.033 g, 0.031 mmol, 0.15 equiv) was added, after the degazation the solution was filled with H₂. The reaction mixture was stirred at room temperature under H₂ (1 atm) for 20 h. The solution was filtered through Celite®, the solvent was removed in vacuum, and product was dried in vacuum overnight. Yield 0.1 g, 99%. ¹H NMR (300 MHz, CDCl₃, δ): 1.19 (3H, t, J=7.2 Hz, CH₃CH₂), 1.28-1.39 (3H+2H, t+m, J=7.2 Hz), 1.64-1.81 (8H, m), 2.40 (2H, m, CH₂), 2.66 (2H, t, J=7.2 Hz, CH₂), 3.00 (2H, m, CH₂), 3.37 (2H, q, J=7.2 Hz, NCH₂CH₃), 3.50 (2H, q, J=7.2 Hz, NCH₂CH₃), 3.83 (2H, t, J=6.3 Hz, CH₂NH), 7.35-7.39 (2H, in, ArH), 7.61 (1H, t, J=7.2 Hz. ArH), 7.92 (1H, d, J=8.4 Hz, ArH), 8.07 (1H, d, J=9 Hz, ArH), 8.37 (1H, d, J=5.1 Hz, ArH). ¹³C NMR (100 MHz, CDCl₃, δ): 13.44, 14.36, 20.79, 21.96, 23.64, 25, 61, 27.58, 28.27, 29.17, 30.29, 42.75, 43.19, 47.98, 111.42, 114.74, 115.95, 120.52, 124.48, 125.33, 128.53, 129.67, 132.65, 138.88, 146.11, 147.75, 150.45, 151.35, 152.52, 155.99. MS m/z (ESI) calcd. for C₂₉H₃₆N₅O₂ [M+H]⁺, 486.28. Found, 486.2.

### N′,3-dihydroxy-4-{5-[(1,2,3,4-tetrahydroacridin-9-yl)amino]pentyl}pyridine-2-carboximidamide (6)

Compound 6 was synthesized accordingly to the general procedure 2 using 21 (0.03 g, 0.06177 mmol, 1 equiv), NH₂OH.HCl (0.129 g, 1.85 mmol, 30 equiv.), pyridine (0.147 g, 0.45 mL, 5.56 mmol, 30 equiv) and 2 mL of ethanol. The product was purified by preparative HPLC chromatography (CH₃CN, H₂O, TFA 0.1%, gradient H₂O from 95% to 5%). Yield 5 mg, 20%. ¹H NMR (300 MHz, MeOD-D₄, δ): 1.37-1.47 (2H, m), 1.64-1.73 (2H, qui, J=7.3 Hz, CH₂CH₂), 1.80-1.95 (2H+4H, qui+m, J=7.3 Hz, CH₂CH₂), 2.60 (2H, m, CH₂), 2.67 (2H, t, J=7.3 Hz, CH₂), 2.99 (2H, m, CH₂), 3.94 (2H, t, J=7 Hz, CH₂), 7.05 (1H, d, J=4.7 Hz, ArH), 7.53-7.59 (1H, td, J=7 Hz, J=1.4 Hz, ArH), 7.73-7.75 (1H, m, ArH), 7.82-7.86 (1H, m, ArH), 7.90 (1H, d, J=4.7 Hz, ArH), 8.34 (1H, d, J=8.6 Hz, ArH). ¹³C NMR (100 MHz, MeOD-D₄, δ): 21.98, 23.08, 24.94, 26.91, 29.12, 29.41, 29.82, 31.17, 112.97, 117.20, 120.29, 126.45, 126.58, 126.75, 133.02, 134.22, 139.81, 139.89, 140.21, 151.82, 154.82, 154.15, 155.31, 158.19. MS m/z (ESI) calcd. for C₂₄H₃₀N₅O₂ [M+H]⁺, 420.23. Found, 420.2.

### 1.2—Synthesis of Compound 7

### 3-(Benzyloxy)-6-{5-[(1,2,3,4-tetrahydroacridin-9-yl)amino]pent-1-yn-1-yl}pyridine-2-carbonitrile (23)

Compound 23 was obtained accordingly to the general procedure 1 for Sonogashira coupling reaction using 22 (0.099 g, 0.000345 mol, 1 equiv), 19 (0.100 g, 0.0003797 mol, 1.1 equiv), Pd(PPh₃)₄ (0.031 g, 0.0000345 mol, 0.1 equiv), CuI (0.013 g, 0.000069 mol, 0.2 equiv), Et₃N (3 mL), and THF (3 mL). The crude product was purified by the column chromatography (silica gel, gradient from ethyl acetate to ethyl acetate/MeOH=8/2, R_{f}=0.28). Yield 0.140 g, 86%. ¹H NMR (400 MHz, CD₃OD, δ): 1.89-1.92 (4H, m, CH₂); 2.09 (2H, qui, J=6.7 Hz, CH₂); 2.59 (2H, t, J=6.7 Hz, CH₂); 2.75 (2H, t, J=5.5 Hz, CH₂); 2.97 (2H, t, J=5.8 Hz, CH₂); 4.08 (2H, t, J=6.7 Hz, CH₂); 5.33 (2H, s, OCH₂); 7.34-7.52 (7H, m, ArH); 7.66 (1H, d, J=8.9 Hz, ArH); 7.70-7.72 (2H, m, ArH); 8.36 (1H, d, J=8.7 Hz, ArH). ¹³C NMR (100 MHz, CD₃OD, δ): 17.30; 22.16; 23.23; 25.39; 29.79; 30.28; 30.43; 30.78; 48.16; 72.44; 90.78; 117.96; 119.83; 121.60; 123.59; 125.98; 126.21; 128.71; 129.68; 129.84; 132.84; 133.21; 151.89.

### 3-Hydroxy-6-{5-[(1,2,3,4-tetrahydroacridin-9-yl)amino]pentyl}pyridine-2-carbonitrile (24)

Compound 24 was obtained accordingly to the general procedure 3 for the one-pot reduction of the triple bond and O-debenzylation using 23 (0.10 g, 0.0002116 mol, 1 equiv), Pd(OH)₂/C (20%, 0.089 g, 0.0001269 mol, 0.6 equiv), and 2 mL of MeOH. Yield 0.080 g, 80%. R_{f}=0.34 (CH₂Cl₂/MeOH=10/1). ¹H NMR (300 MHz, CD₃OD, δ): 1.35-1.45 (2H, m, CH₂); 1.65-1.75 (2H, m, Cl-h); 1.79-1.89 (2H, m, CH₂); 1.94 (4H, m, CH₂); 2.63-2.68 (4H, m, CH₂); 3.02 (2H, m, CH₂); 3.93 (2H, t, J=7 Hz, CH₂); 7.19-7.27 (2H, dd, J=8.7 Hz, ArH); 7.53-7.58 (1H, m, ArH); 7.76-7.85 (2H, m, ArH); 8.34 (1H, d, J=8.7 Hz, ArH).

### N′,3-dihydroxy-6-{5-[(1,2,3,4-tetrahydroacridin-9-yl)amino]pentyl}pyridine-2-carboximidamide (7)

NH₂OH.HCl (0.015 g, 0.0002098 mol, 5 equiv) and Na₂CO₃ (0.011 g, 0.0001049 mol) were stirred in 0.2 mL of water. Then this solution was added to nitrile 24 (0.02 g, 0.00004196 mol, 1 equiv) and the reaction mixture was stirred overnight. The solvents were evaporated, and the crude product was dried in vacuum. The purification by the column chromatography (silica gel, CH₂Cl₂/MeOH=10/1) resulted in pure amidoxime 7 with 65% yield. R_{f}=0.42 (CH₂Cl₂/MeOH=9/1). ¹H NMR (500 MHz, CD₃OD, δ): 1.36-1.42 (2H, m, CH₂), 1.74-1.84 (2H+2H, m+m, CH₂), 1.89-1.94 (4H, m, CH₂), 2.57 (2H, m, CH₂), 2.70 (2H, t, J=7.1 Hz, CH₂), 2.99 (2H, m, CH₂), 3.89 (2H, t, J=6.9 Hz, CH₂), 6.99 (1H, d, J=8.4 Hz, ArH), 7.03 (1H, d, J=8.4 Hz, ArH); 7.51-7.54 (1H, m, ArH); 7.74-7.76 (1H, m, ArH); 7.79-7.82 (1H, m, ArH); 8.30 (1H, d, J=8.7 Hz, ArH). ¹³C NMR (125 MHz, CD₃OD, δ): 22.11; 23.13; 25.02; 26.73; 29.77; 29.86; 31.23; 37.51; 113.26; 117.51; 120.88; 125.58; 125.64; 126.30; 126.44; 132.53; 133.86; 140.49; 152.34; 152.49; 153.60; 155.10; 157.75. HRMS m/z (ESI) calcd. for C₂₄H₃₀N₅O₂ [M+H]⁺, 420.2399. Found, 420.2433.

### 1.3—Synthesis of Compound 8

### 2-Cyano-4-formylpyridine-3-yl N,N-diethylcarbamate (25)

To the solution of 17 (0.5 g, 2.28 mmol, 1 equiv) in 20 mL of THF at −78° C. the solution of n-BuLi (2.5M, in hexanes, 1.37 mL, 3.42 mmol, 1.5 equv) was added dropwise. After 30 min of stirring at −78° C. solution of DMF (1.17 g, 15.9 mmol, 7 equiv) in 5 mL of THF was added. The reaction mixture was stirred at −78° C. for 2.5 h. The reaction was quenched with 10 mL of saturated solution of NH₄Cl, the organic phase was separated, and the product was extracted from the aqueous phase with ethyl acetate. The organic phases were combined and dried over Na₂SO₄. After removal of the solvent under reduced pressure the crude was subjected to the column chromatography on Al₂O₃ basic (CHCl₃/MeOH/Et₃N=100/0.5/0.5, R_{f}=0.32). Yield 0.3 g, 53%. ¹H NMR (500 MHz, CDCl₃, δ): 1.23 (3H, t, J=7.1 Hz, CH₃CH₂N), 1.35 (3H, t, J=7.1 Hz, CH₃CH₂N), 3.40 (2H, q, J=7.1 Hz, NCH₂CH), 3.56 (2H, q, J=7.1 Hz, NCH₂CH₃), 7.89 (1H, d, J=4.7 Hz, ArH), 8.72 (1H, d, J=4.7 Hz, ArH), 10.08 (1H, s, CHO). ¹³C NMR (125 MHz, CDCl₃, δ): 13.19; 14.20; 42.74; 43.28; 113.89; 125.42; 130.79; 135.78; 148.65; 150.33; 151.83; 185.78.

### 2-Cyano-4-{[methyl({2-[(1,2,3,4-tetrahydroacridin-9-yl)amino]ethyl}) amino]methyl}pyridin-3-yl N,N-diethylcarbamate (28)

Amine 26 (0.2 g, 0.78 mmol, 1.05 equiv) and aldehyde 25 (0.184 g, 0.744 mmol, 1 equiv) were stirred in 3 mL of THF for 10 min, then NaBH(OAc)₃ (0.232 g, 1.09 mmol, 1.4 equiv) was added. The reaction mixture was stirred at room temperature for 2.5 h, then saturated solution of NaHCO₃ (0.2 mL) was added. The solvent was evaporated and the residue was purified by the column chromatography (silica gel, CHC₃/MeOH/Et₃N=100/0.5/0.5, R_{f}=0.37). Yield of 28 0.272 g, 75%. ¹H NMR (400 MHz, CDCl₃, δ): 1.18 (3H, t, J=7 Hz, CH₃CH₂N), 1.29 (3H, t, J=7 Hz, CH₃CH₂N), 1.83-1.85 (4H, m, CH₂CH₂CH₂CH₂), 2.24 (3H, s, CH₃N), 2.62 (2H, t, J=5.8 Hz, CH₂CH₂), 2.66 (2H, t, J=5.6 Hz, CH₂CH₂), 3.01 (2H, t, J=5.6 Hz, CH₂CH₂); 3.34 (2H, q, J=7.2 Hz, NCH₂CH₃), 3.47 (2H, q, J=7.2 Hz, NCH₂CH₃), 3.55-3.59 (2H+2H, s+m, ArCH₂NMe+CH₂N), 4.93 (1H, br. s., NH); 7.26-7.29 (1H, m, ArH); 7.48-7.52 (1H, m, ArH); 7.63 (1H, d, J=4.8 Hz, ArH); 7.88-7.91 (1H, m, ArH); 8.45 (1H, d, J=4.8 Hz, ArH). ¹³C NMR (100 MHz, CDCl₃, δ): 13.33; 14.35; 22.70; 23.05; 24.93; 33.55; 42.13; 42.58; 43.05; 46.06; 55.31; 58.04; 114.62; 115.65; 119.96; 122.83; 123.90; 127.61; 128.16; 128.78; 129.62; 143.57; 146.73; 147.86; 150.16; 151.19; 151.91; 157.99. HRMS m/z (ESI) calcd. for C₂₈H₃₅N₆O₂ [M+H]⁺, 487.28214. Found, 487.29518.

### 2-cyano-4-{[methyl({3-[(1,2,3,4-tetrahydroacridin-9-yl)amino]propyl})amino]methyl}pyridin-3-yl N,N-diethylcarbamate (29)

Amine 27 (0.060 g, 0.000222 mol, 1.1 equiv) and aldehyde 25 (0.05 g, 0.000202 mol, 1 equiv) were stirred in 1 mL of THF for 10 min, then NaBH(OAc)₃ (0.060 g, 0.0002827 mmol, 1.4 equiv) was added. The reaction mixture was stirred at room temperature for 2.5 h, then saturated solution of NaHCO₃ (0.1 mL) was added. The solvent was evaporated and the residue was purified by the column chromatography (silica gel, CHCl₃/MeOH/Et₃N=100/01/0.5, R_{f}=0.57). Yield of 29 0.100 g, 99%. ¹H NMR (400 MHz, CDCl₃, δ): 1.20 (3H, t, J=7.2 Hz, CH₃CH₂), 1.30 (3H, t, J=7.2 Hz); 1.80-1.89 (2H+4H, qui+m, J=6.7 Hz, CH₂), 2.19 (3H, s, CH₃N), 2.46 (2H, t, J=6.7 Hz), 2.59 (2H, t, J=5.9 Hz), 3.03 (2H, t, J=5.9 Hz), 3.34-3.39 (2H, q, J=7.2 Hz), 3.45-3.50 (2H+2H, s+quart, J=7.2), 3.55 (2H, t, J=6.5 Hz), 7.26-7.30 (1H, m, J=6.8 Hz, J=1.3 Hz), 7.50-7.54 (1H, m, J=6.8 Hz, J=1.3 Hz), 7.56 (1H, d, J=4.9 Hz), 7.87-7.91 (2H, m), 8.38 (1H, d, J=4.9 Hz). ¹³C NMR (100 MHz, CDCl₃, δ): 13.39; 14.36; 22.81; 23.13; 25.02; 29.04; 33.85; 42.59; 42.75; 43.05; 47.36; 55.42; 55.72; 114.71; 115.83; 120.10; 122.84; 123.84; 127.84; 128.52; 128.63; 129.54; 144.01; 147.13; 147.84; 150.16; 150.92; 152.03; 158.24.

### (Z)—N′,3-dihydroxy-4-{[methyl({2-[(1,2,3,4-tetrahydroacridin-9-yl)amino]ethyl})amino]methyl}pyridine-2-carboximidamide (8)

Compound 8 was synthesized accordingly to the general procedure 2 using 28 (0.03 g, 0.0000616 mol), NH₂OH.HCl (0.128 g, 0.001849 mol, 30 equiv), pyridine (0.149 mL, 0.001849 mol, 30 equiv). The crude product was purified by preparative HPLC (CH₃CN, H₂O, 0.01 M HCOONH₄, NH₄OH, pH 9, gradient H₂O from 95% to 5%). Yield 8 mg, 32%. ¹H NMR (400 MHz, CD₃OD, δ): 1.93-1.96 (4H, m), 2.63 (2H, t, J=5.5. Hz), 3.01 (3H, s, CH₃N), 3.03 (2H, m), 3.55 (2H, t, J=5.1 Hz), 4.31-4.33 (2H+2H, t+s, CH₂), 7.23 (1H, d, J=4.6 Hz), 7.54-7.57 (1H, m, ArH), 7.76-7.78 (2H, m), 7.83-7.87 (1H, m), 8.21 (1H, d, J=8.8 Hz). ¹³C NMR (100 MHz, CD₃OD, δ): 17.42; 18.85; 21.84; 22.89; 24.86; 29.38; 43.61; 55.97; 56.21; 57.00; 117.05; 120.51; 126.01; 126.72; 127.76; 133.62; 134.21; 139.72; 140.09; 154.94; 157.11. HRMS (ESI, m/z): calcd. for C₂₃H₂₉N₆O₂ [M+H]⁺ 421.23519. Found, 421.23561.

### 1.4—Synthesis of Compound 9

### (Z)—N′,3-dihydroxy-4-{[methyl({3-[(1,2,3,4-tetrahydroacridin-9-yl)amino]propyl})amino]methyl}pyridine-2-carboximidamide (9)

Compound 9 was obtained accordingly to the general procedure 2 using 29 (0.050 g, 0.00009986 mol, 1 equiv), NH₂OH.HCl (0.208 g, 0.002995 mol, 30 equiv), pyridine (0.24 mL, 0.002996 mol, 30 equiv). The crude product was purified by preparative HPLC (CH₃CN, H₂O, 0.01 M HCOONH₄, NH₄OH, pH 9, gradient H₂O from 95% to 5%). Yield 17 mg, 37%. ¹H NMR (400 MHz, CD₃OD, δ): 1.93-1.96 (4H, m); 2.28-2.36 (2H, m, CH₂); 2.60 (2H, m, CH₂); 2.93 (3H, s, CH₃N); 3.04 (2H, m, CH₂); 3.25-3.29 (2H, m, CH₂); 4.04 (2H, t, J=6.7 Hz, CH₂); 4.41 (2H, s, CH₂); 7.32 (1H, d, J=4.8 Hz, ArH), 7.56-7.60 (1H, m, ArH), 7.78-7.80 (1H, dd, J=8.5 Hz, J=1 Hz, ArH); 7.84-7.89 (1H, m, ArH); 8.03 (1H, d, J=4.8 Hz, ArH); 8.31 (1H, d, J=8.5 Hz, ArH). ¹³C NMR (100 MHz, CD₃OD, δ): 21.86; 23.03; 25.20; 26.27; 29.48; 41.91; 45.68; 54.23; 55.19; 113.52; 117.32; 120.59; 126.11; 126.55; 126.91; 128.08; 134.31; 139.79; 140.45; 152.51; 155.07; 155.33; 157.80. HRMS (ESI, m/z): calcd. for C₂₄H₃₁N₆O₂ [M+H]⁻ 435.25084. Found, 435.25099.

### 1.5—Synthesis of Compound 10

### Methyl 3-hydroxy-4-{5-[(1,2,3,4-tetrahydroacridin-9-yl)amino]pentyl}pyridine-2-carboxylate (30)

To the solution of 21 (0.140 g, 0.000288 mol) in dry MeOH (2 mL) 0.3 mL of H₂SO₄ (98%) was added dropwise. The reaction mixture was refluxed during 24 h. After cooling to the room temperature the reaction mixture was quenched with concentrated solution of Na₂CO₃. The product was extracted with CH₂Cl₂ and purified by the column chromatography (CH₂Cl₂/MeOH=10/1) to give 30 with 42% yield. R_{f}=0.45 (CH₂Cl₂/MeOH=10/1). ¹H NMR (500 MHz, CDCl₃, δ): 1.39-1.46 (2H, m, CH₂); 1.64 (2H, qui, J=7.7 Hz, CH₂); 1.74-1.83 (6H, m, CH₂); 2.61 (2H, t, J=6.1 Hz, CH₂); 2.64 (2H, t, J=7.7 Hz, CH₂; 3.05 (2H, t, J=5.7 Hz); 3.69 (2H, t, J=7.1 Hz); 3.98 (3H, s, CH₃O); 5.49 (1H, br.s., NH); 7.19 (1H, J=4.4 Hz, ArH); 7.29-7.33 (1H, m, ArH); 7.51-7.54 (1H, m, ArH); 8.03-8.05 (2H, m, ArH); 8.09 (1H, d, J=4.4 Hz, ArH). ¹³C NMR (125 MHz, CDCl₃, δ): 21.68; 22.52; 24.38; 26.55; 28.23; 29.01; 30.88; 31.08; 48.72; 53.29; 113.25; 117.78; 123.88; 124.08; 124.53; 129.14; 129.48; 130.68; 140.60; 141.22; 142.51; 153.69; 154.24; 157.64; 170.44.

### 3-Hydroxy-4-{5-[(1,2,3,4-tetrahydroacridin-9-yl)amino]pentyl}pyridine-2-carbaldehyde (31)

Aldehyde 31 was obtained from methyl ester 30 accordingly to the general procedure 4 and purified by the column chromatography (silica gel, ethyl acetate/MeOH=10/1) with yield 33% (over three steps). R_{f}=0.35 (ethyl acetate/MeOH=11/3). ¹H NMR (300 MHz, CDCl₃, δ): 1.40-1.50 (2H, m, CH₂); 1.62-1.77 (4H, m, CH₂); 1.84-1.91 (4H, m, CH₂); 2.64-2.71 (4H, m, CH₂); 3.07 (2H, t, CH₂); 3.54 (2H, t, J=7.2 Hz, CH₂); 7.20-7.23 (1H, m, ArH); 7.30-7.35 (1H, td, J=7.3 Hz, J=0.9 Hz, ArH); 7.52-7.57 (1H, td, J=6.9 Hz, J=0.9 Hz); 7.93-7.98 (2H, m, ArH); 8.21 (1H, d, J=4.5 Hz, ArH); 10.04 (1H, s, CHO).

### 2-[(hydroxyimino)methyl]-4-{5-[(1,2,3,4-tetrahydroacridin-9-yl)amino]pentyl}pyridin-3-ol (10)

Compound 10 was obtained accordingly to the general procedure 5 using 31 (0.015 g, 0.03851 mmol, 1 equiv), NH₂OH.HCl (0.004 g, 0.05777 mmol, 1.5 equiv), CH₃COONa (0.005 g, 0.05777 mmol, 1.5 equiv), and 1 mL of ethanol. The crude product was purified by the column chromatography (silica gel, ethyl acetate/MeOH=10/1) to give 10 with 51% yield. R_{f}=0.44 (ethyl acetate/MeOH=2/1). ¹H NMR (300 MHz, CD₃OD, δ): 1.37-1.47 (2H, m, CH₂); 1.62-1.80 (2H+2H, m+m, CH₂); 1.90-1.93 (4H, m, CH₂); 2.66-2.71 (4H, m, CH₂); 2.99 (2H, m, CH₂); 3.71 (2H, t, J=6.9 Hz, CH₂); 7.08 (1H, d, J 4.8 Hz, ArH); 7.40-7.46 (1H, m, ArH); 7.64-7.69 (1H, m, ArH); 7.74-7.77 (1H, m, ArH); 7.96 (1H, d, J=4.8 Hz, ArH); 8.18 (1H, d, J=8.7 Hz, ArH); 8.26 (1H, s, CH═N). ¹³C NMR (125 MHz, CD₃OD δ): 23.17; 23.83; 25.82; 27.49; 29.39; 29.96; 30.92; 31.81; 32.53; 115.45; 119.88; 125.29; 125.43; 126.48; 131.48; 136.41; 140.74; 141.65; 153.01; 154.31; 155.14; 156.52. HRMS (ESI, m/z): calcd. for C₂₄H₂₉N₄O₂ [M+H]⁺ 405.2290. Found, 405.2301.

### 1.6—Synthesis of Compound 11

### Methyl 3-(benzyloxy)-6-{5-[(1,2,3,4-tetrahydroacridin-9-yl)amino]pent-1-yn-1-yl}-pyridine-2-carboxylate (35)

Compound 35 was obtained accordingly to the general procedure 1 for Sonogashira coupling reaction using 32 (0.147 g, 0.4556 mmol, 1 equiv), 19 (0.132 g, 0.502 mmol, 1.1 equiv), Pd(PPh₃)₄ (0.041 g, 0.04556 mmol, 0.1 equiv), CuI (0.017 g, 0.09112 mmol, 0.2 equiv), Et₃N (3 mL), and THF (6 mL). The crude product was purified by the column chromatography (silica gel, gradient from ethyl acetate to ethyl acetate/MeOH=8/2, Rt=0.24). Yield 180 mg, 78%. ¹H NMR (300 MHz, CDCl₃, δ): 1.76-1.84 (4H, m), 1.87-1.97 (2H, qui, J=6.7 Hz), 2.49 (2H, t, J=6.7 Hz), 2.64 (2H, m), 3.03 (2H, m), 3.67 (2H, t, J=6.9 Hz), 3.88 (3H, s, CH₃O), 5.14 (2H, s, PhCH₂O), 7.18-7.38 (8H, m, ArH), 7.48 (1H, t, J=7.6 Hz), 7.90-7.93 (2H, m). ¹³C NMR (100 MHz, CDCl₃, δ): 17.29; 22.79; 23.13; 25.05; 29.98; 33.79; 48.42; 52.88; 71.10; 80.64; 88.84; 120.24; 121.96; 122.93; 124.19; 127.12; 128.37; 128.47; 128.63; 128.75; 128.83; 128.95; 130.11; 132.24; 132.34; 135.23; 135.66; 146.91; 150.97; 153.20; 158.23; 164.99.

### Methyl 3-hydroxy-6-{5-[(1,2,3,4-tetrahydroacridin-9-yl)amino]pentyl}pyridine-2-carboxylate (38)

Compound 38 was obtained accordingly to the general procedure 3 for the reduction of the triple bond and O-debenzylation using 35 (0.180 g, 0.355 mmol), Pd(OH)₂/C (20%, 0.075 g, 0.1066 mmol, 0.3 equiv), and 2.5 mL of MeOH. Yield 0.140 g, 94%. R_{f}=0.34 (CH₂Cl₂/MeOH=10/1). ¹H NMR (300 MHz, CDCl₃, δ): 1.40-1.50 (2H, m), 1.67-1.78 (4H, m), 1.87-1.92 (4H, m), 2.63 (2H, m), 2.78 (2H, t, J=7.9 Hz), 3.11 (2H, m), 3.56 (2H, t, J=7.2 Hz), 4.00 (3H, s, CH₃O), 7.23 (1H, d, J=8.6 Hz), 7.28 (1H, d, J=8.6 Hz), 7.31-7.36 (1H, m), 7.54-7.59 (1H, m), 7.95 (1H, d, J=8 Hz), 8.05 (1H, d, J=8.6 Hz). ¹³C NMR (100 MHz, CDCl₃, δ): 20.87; 22.11; 23.78; 26.38; 28.60; 29.62; 31.09; 37.02; 48.61; 53.55; 110.88; 115.99; 121.66; 124.19; 125.49; 127.88; 129.65; 132.63; 139.27; 152.03; 153.45; 155.55. HRMS (ESI, m/z): calcd. for C₂₅H₃₀N₃O₃ [M+H]⁺, 420.22871. Found, 420.22836.

### 3-Hydroxy-6-{5-[(1,2,3,4-tetrahydroacridin-9-yl)amino]pentyl}pyridine-2-carbaldehyde (41)

Compound 41 was obtained by the general procedure 4 from 38 with yield 37% (over three steps). R_{f}=0.23 (ethyl acetate/MeOH=10/3). ¹H NMR (400 MHz, CDCl₃, δ): 1.40-1.48 (2H, m), 1.67-1.79 (4H, m), 1.85-1.90 (4H, m), 2.64 (2H, m), 2.76 (2H, t, J=7.7 Hz), 3.05 (2H, m), 3.52 (2H, t, J=7.3 Hz), 7.23 (1H, d, J=8.7 Hz), 7.25 (1H, d, J=8.7 Hz), 7.29-7.33 (1H, m, ArH), 7.51-7.55 (1H, m, ArH), 7.92-7.95 (2H, m), 9.99 (1H, s, CHO). ¹³C NMR (100 MHz, CDCl₃, δ): 22.73; 23.11; 24.88; 26.70; 29.48; 31.76; 33.48; 37.28; 49.45; 115.48; 123.10; 124.02; 126.64; 127.99; 129.01; 129.88; 131.06; 135.95; 151.45; 154.66; 157.23; 157.88; 198.83. HRMS (ESI, m/z): calcd. for [M+H]⁺, C₂₄H₂₈N₃O₂, 390.21815. Found, 390.21892.

### 2-[(1E)-(hydroxyimino)methyl]-6-{5-[(1,2,3,4-tetrahydroacridin-9-yl)amino]pentyl}pyridin-3-ol (11)

Compound 11 was obtained accordingly to the general procedure 5 using 41 (0.050 g, 0.00012837 mol, 1 equiv), NH₂OH.HCl (0.013 g, 0.00019256 mol, 1.5 equiv), CH₃COONa (0.016 g, 0.00019256 mol, 1.5 equiv), and 1.2 mL of ethanol. After evaporation of the solvent and purification by column chromatography (ethyl acetate/MeOH-8/2) product was obtained with yield 55%. R_{f}=0.44 (ethyl acetate/MeOH=2/1). ¹H NMR (400 MHz, CD₃OD, δ): 1.37-1.44 (2H, m, CH₂), 1.68-1.76 (2H, qui, J=7.4 Hz, CH₂), 1.77-1.85 (2H, qui, J=7.4 Hz, CH₂), 1.90-1.94 (4H, qui, J=3.3 Hz, CH₂), 2.63 (2H, m, CH₂), 2.70 (2H, t, J=7.4 Hz, CH₂), 3.00 (2H, m, CH₂), 3.87 (2H, t, J=6.9 Hz, CH₂), 7.08 (1H, d, J=8.4 Hz, ArH), 7.20 (1H, d, J=8.4 Hz, ArH), 7.50-7.54 (1H, m, ArH), 7.76-7.81 (2H, m, ArH), 8.17 (1H, s, N═CH), 8.30 (1H, d, J=8.7 Hz). ¹³C NMR (100 MHz, CD₃OD, δ): 22.31; 23.30; 25.25; 27.05; 30.30; 30.53; 30.91; 31.42; 37.56; 113.66; 117.95; 118.57; 121.70; 125.51; 126.08; 126.18; 133.42; 136.35; 152.90; 153.85; 154.49. MS m/z (ESI) calcd. for C₂₄H₂₉N₄O₂ [M+H]⁺, 405.22. Found, 405.2.

### 1.7—Synthesis of Compound 12

### Methyl 3-(benzyloxy)-6-{4-[(1,2,3,4-tetrahydroacridin-9-yl)amino]but-1-yn-1-yl}pyridine-2-carboxylate (36)

Compound 36 was obtained accordingly to the general procedure 1 for Sonogashira coupling reaction using 32 (0.292 g, 0.0009078 mol, 1 equiv), 34 (0.250 g, 0.0009986 mol, 1.1 equiv), Pd(PPh₃)₄ (0.081 g, 0.00009078 mol, 0.1 equiv), CuI (0.035 g, 0.00018156 mol, 0.2 equiv), Et₃N (6 mL), and THF (12 mL). The crude product was purified by the column chromatography (silica gel, gradient from ethyl acetate/MeOH 10/1 to ethyl acetate/MeOH=8/2), R_{f}=0.43 (ethyl acetate/MeOH=4/1). Yield 0.440 g, 98%. ¹H NMR (400 MHz. CDCl₃, δ): 1.82-1.89 (4H, m), 2.67 (2H, t, J=6.3 Hz), 2.79 (2H, t, J=5.9 Hz), 3.06 (2H, t, J=5.9 Hz), 3.70-3.75 (2H, dt, J=6.4 Hz), 3.95 (3H, s), 4.48 (1H, t, J=6.4 Hz, NH), 5.19 (2H, s, OCH₂Ph), 7.25 (1H, d, J=8.8 Hz), 7.28 (2H, d, J=8.8 Hz), 7.31-7.42 (6H, m, ArH), 7.52-7.56 (1H, m), 7.97 (2H, t, ArH, J=8.3 Hz). ¹³C NMR (100 MHz, CDCl₃, δ): 22.10; 22.59; 22.99; 24.93; 29.89; 33.29; 47.34; 52.93; 71.10; 81.98; 86.71; 116.94; 120.15; 121.92; 123.00; 124.56; 127.14; 127.66; 128.51; 128.97; 129.30; 130.09; 134.74; 135.59; 140.67; 146.04; 151.08; 153.37; 157.78; 164.98. HRMS (ESI, m/z): calcd. for C₃₁H₃₀N₃O₃ [M+H]⁺, 492.22871. Found, 492.22851.

### Methyl 3-hydroxy-6-{4-[(1,2,3,4-tetrahydroacridin-9-yl)amino]butyl}pyridine-2-carboxylate (39)

Compound 39 was obtained accordingly to the general procedure 3 using 36 (0.430 g, 0.0008747 mol), Pd(OH)₂/C (20%, 0.184 g, 0.0002624 mol, 0.3 equiv), and 3 mL of MeOH. Yield 0.340 g, 96%. R_{f}=0.43 (CH₂Cl₂/MeOH=10/1). ¹H NMR (300 MHz, CD₃OD, δ): 1.67-1.74 (4H, m), 1.87-1.91 (4H, m), 2.68 (2H, t, J=5.3 Hz), 2.72 (2H, t, J=7 Hz), 2.96 (2H, t, J=5.5 Hz), 3.62 (2H, t, J=6.7 Hz), 3.96 (3H, s, CH₃O), 7.23 (1H, d, J=8.6 Hz), 7.27 (2H, d, J=8.6 Hz), 7.34-7.40 (1H, m), 7.57-7.62 (1H, m), 7.74 (1H, d, J=8.4 Hz), 8.10 (1H, d, J=8.4 Hz). ¹³C NMR (100 MHz, CD₃OD, δ): 23.25; 23.87; 25.96; 28.22; 31.31; 32.84; 37.27; 53.34; 115.73; 120.15; 125.07; 125.29; 125.74; 128.52; 130.46; 130.65; 131.09; 145.48; 154.49; 154.54; 156.94; 158.65; 170.62. HRMS (ESI, m/z): calcd. for C₂₄H₂₈N₃O₃ [M+H]⁺, 406.21306. Found, 406.21307.

### 3-Hydroxy-6-{4-[(1,2,3,4-tetrahydroacridin-9-yl)amino]butyl}pyridine-2-carbaldehyde (42)

Compound 42 was obtained from 39 accordingly to the general procedure 4 after purification by the column chromatography (silica gel, ethyl acetate/MeOH=8/2) with yield 54% (over 3 steps). R_{f}=0.21 (ethyl acetate/MeOH=7/3). ¹H NMR (400 MHz, CDCl₃, δ): 1.66-1.75 (2H, m), 1.78-1.89 (6H, m), 2.67 (2H, m), 2.79 (2H, t, J=7 Hz), 3.04 (2H, m), 3.52 (2H, t, J=7 Hz), 7.20 (1H, d, J=8.6 Hz), 7.24 (1H, d, J=8.6 Hz), 7.31 (1H, td, J=6.8 Hz, J=1 Hz), 7.53 (1H, td, J=6.8 Hz, J=1 Hz), 7.89-7.93 (2H, m), 9.98 (1H, s, CH═O). ¹³C NMR (100 MHz, CDCl₃, δ): 22.89; 23.20; 25.01; 26.96; 31.37; 33.92; 36.98; 49.32; 116.06; 120.29; 122.94; 123.95; 126.69; 128.68; 129.87; 136.00; 147.27; 150.99; 154.29; 157.29; 158.43; 198.74. HRMS (ESI, m/z): calcd. for C₂₃H₂₆N₃O₂ [M+H]⁺, 376.20250. Found, 376.20201.

### 2-[1-(hydroxymino)methyl]-6-{4-[(1,2,3,4-tetrahydroacridin-9-yl)amino]butyl}pyridin-3-ol (12)

Compound 12 was obtained accordingly to the general procedure 5 using 42 (0.090 g, 0.0002397 mol, 1 equiv), NH₂OH.HCl (0.025 g, 0.0003596 mol, 1.5 equiv), CH₃COONa (0.029 g, 0.0003596 mol, 1.5 equiv), and 2.5 mL of ethanol. Compound 12 was purified by the column chromatography (silica gel, CH₂Cl₂/MeOH=8/2) with yield 55%. R_{f}=0.6 (ethyl acetate/MeOH=2/1). ¹H NMR (400 MHz, CD₃OD, δ): 1.80-1.83 (4H, m, CH₂CH₂CH₂CH₂), 1.90-1.95 (4H, m, CH₂CH₂CH₂CH₂), 2.62 (2H, t. J=5.6 Hz, CH₂), 2.71 (2H, t, J=6.4 Hz, CH₂), 2.98 (2H, m, CH₂), 3.87 (2H, t, J=5.6 Hz, CH₂), 7.04 (1H, d, J=8.4 Hz, ArH), 7.09 (1H, d, J=8.4 Hz); 7.47-7.51 (1H, m, ArH), 7.71-7.79 (2H, m, ArH), 8.08 (1H, s, CH═N), 8.24 (1H, d, J=8.8 Hz). ¹³C NMR (100 MHz, CD₃OD, δ): 22.04; 23.09; 25.03; 27.55; 29.84; 30.60; 37.11; 113.35; 117.54; 121.08; 125.43; 125.83; 126.12; 126.19; 133.51; 136.24; 140.59; 152.44; 152.75; 153.65; 154.12; 157.25. HRMS (ESI, m/z): calcd. for C₂₃H₂₇N₄O₂ [M+H]⁺ 391.21340. Found, 391.21337.

### 1.8—Synthesis of Compound 13

### Methyl 3-(benzyloxy)-{5-[(1,2,3,4-tetrahydroacridin-9-yl)amino]pent-1-yn-1-yl}pyridine-2-carboxylate (37)

Compound 37 was obtained accordingly to the general procedure 1 using 33 (0.333 g, 0.001035 mol, 1 equiv), 34 (0.300 g, 0.001139 mol, 1.1 equiv), Pd(PPh₃)₄ (0.093 g, 0.0001035 mol, 0.1 equiv), CuI (0.039 g, 0.0002071 mol, 0.2 equiv), Et₃N (6 mL), and THF (12 mL). The crude product was purified by the column chromatography (silica gel, CH₂Cl₂/MeOH=9/1), R_{f}=0.52 (CH₂Cl₂/MeOH=8/2). Yield 87%. ¹H NMR (300 MHz, CDCl₃, δ): 1.77-1.79 (4H, m, CH₂), 2.14-2.24 (2H, m, CH₂), 2.63 (2H, t, J=6.7 Hz), 2.69 (2H, t, J=5.1 Hz), 3.21 (2H, t, J=5.1 Hz), 3.94 (3H, s, OCH₃), 4.09-4.15 (2H, m), 5.15 (2H, s, OCH₂), 6.68 (1H, br.s., NH), 7.28-7.43 (7H, m, ArH), 7.55-7.60 (1H, m, ArH), 8.19 (1H, d, J=0.9 Hz, ArH), 8.27 (1H, d, J=8.6 Hz), 8.43 (1H, d, J=8.6 Hz). ¹³C NMR (100 MHz, CDC₃, δ): 17.32; 20.84; 22.11; 24.86; 28.81; 29.39; 47.19; 52.79; 70.99; 78.30; 94.74; 111.82; 116.19; 120.58; 124.25; 124.70; 125.28; 127.04; 128.32; 128.82; 132.27; 133.87; 134.83; 135.61; 137.82; 138.98; 143.64; 151.33; 154.26; 155.82; 164.83.

### Methyl 3-hydroxy-5-{5-[(1,2,3,4-tetrahydroacridin-9-yl)amino]pentyl}pyridine-2-carboxylate (40)

Compound 40 was synthesized accordingly to the general procedure 3 using 37 (0.470 g, 0.0009277 mol, 1 equiv), Pd(OH)₂/C (20%, 0.195 g, 0.0002783 mol, 0.3 equiv), and 4 mL of MeOH. Yield 0.344 g, 89%. R_{f}=0.29 (CH₂Cl₂/MeOH=10/1). ¹H NMR (400 MHz, CDCl₃, δ): 1.45-1.53 (2H, m, CH₂), 1.69-1.76 (2H, m, CH₂), 1.81-1.91 (6H, m, CH₂), 2.58 (2H, t, J=6 Hz, CH₂), 2.68 (2H, t, J=7.5 Hz, CH₂), 3.31 (2H, t, J=6 Hz, CH₂), 3.90-3.95 (2H, m, CH₂), 4.02 (3H, s, OCH₃), 5.90 (1H, br.s., NH), 7.15 (1H, d, J=1.6 Hz, ArH), 7.42 (1H, t, J=7.7 Hz), 7.66 (1H, t. J=7.7 Hz), 8.08 (1H, d, J=1.6 Hz), 8.16 (1H, d, J=8.5 Hz), 8.57 (1H, d, J=8.5 Hz), 10.57 (1H, br.s., OH). ¹³C NMR (100 MHz, CDCl₃, δ): 20.78; 22.05; 24.42; 26.29; 28.54; 30.11; 30.86; 32.79; 48.17; 53.16; 111.24; 115.93; 120.41; 124.70; 125.27; 128.01; 130.71; 132.41; 138.73; 142.25; 145.07; 151.03; 155.88; 158.77; 169.94.

### 3-Hydroxy-5-{5-[(1,2,3,4-tetrahydroacridin-9-yl)amino]pentyl}pyridine-2-carbaldehyde (43)

Compound 43 was obtained from 40 accordingly to the general procedure 4 after purification by the column chromatography (silica gel, CH₂Cl₂/MeOH=9/1) with yield 44% (over 3 steps). R_{f}=0.37 (CH₂Cl₂/MeOH=9/1). ¹H NMR (400 MHz, CDCl₃, δ): 1.37-1.45 (2H, m, CH₂), 1.61-1.70 (4H, m, CH₂), 1.84-1.90 (4H, m, CH₂), 2.62 (2H, t, J=7.7 Hz), 2.65 (2H, t, J=5.6 Hz), 3.03 (2H, t, J=5.6 Hz), 3.43-3.47 (2H, m, CH₂), 7.09 (1H, s, ArH), 7.29-7.33 (1H, m, ArH), 7.49-7.53 (1H, m, ArH), 7.87-7.90 (2H, dd, J=8.3 Hz, J=3.6 Hz), 8.12 (1H, s, ArH), 9.99 (1H, s, CHO). ¹³C NMR (100 MHz, CDCl₃, δ): 22.89; 23.18; 24.99; 26.65; 30.32; 31.69; 33.24; 33.99; 49.36; 116.15; 120.33; 122.86; 123.93; 125.05; 128.60; 128.71; 135.37; 143.43; 145.95; 147.34; 150.87; 158.46; 158.88; 198.13.

### 2-[1-(hydroxyimino)methyl]-5-{5-[(1,2,3,4-tetrahydroacridin-9-yl)amino]pentyl}pyridin-3-ol (13)

Compound 13 was obtained accordingly to the general procedure 5 using 43 (0.090 g, 0.0002397 mol, 1 equiv), NH₂OH.HCl (0.025 g, 0.0003596 mol, 1.5 equiv), CH₃COONa (0.029 g, 0.0003596 mol, 1.5 equiv), and 2.5 mL of ethanol. Compound 13 was purified by preparative HPLC (CH₃CN, H₂O, 0.01M HCOONH₄, NH₄OH, pH 9), gradient H₂O from 95% to 5%) with yield 54%. ¹H NMR (400 MHz, CD₃OD, δ): 1.38-1.46 (2H, m, CH₂); 1.65-1.72 (2H, m, CH₂); 1.80-1.87 (2H, m, C-h); 1.92-1.95 (4H, m, CH₂); 2.61-2.65 (4H, m, CH₂); 2.99 (2H, t, J=6 Hz, CH₂); 3.92 (2H, t, J=7 Hz, CH₂); 7.14 (1H, d, J=1.5 Hz, ArH); 7.53-7.57 (1H, td, J=7 Hz, J=1.4 Hz, ArH); 7.73-7.76 (1H, m, ArH); 7.79-7.84 (1H, m, ArH); 7.91 (1H, d, J=1.5 Hz, ArH); 8.24 (1H, s, CH═N); 8.33 (1H, d, J=8.7 Hz); 8.56 (1H, s, OH). ¹³C NMR (100 MHz, CD₃OD, δ): 22.63; 23.49; 25.45; 27.15; 31.20; 31.39; 31.59; 33.35; 49.25; 114.39; 118.73; 123.17; 125.21; 125.77; 125.86; 127.74; 132.58; 135.42; 141.48; 142.20; 142.87; 152.39; 156.30; 170.45. HRMS (ESI, m/z): calcd. for C₂₄H₂₉N₄₀O₂ [M+H]⁺ 405.22905. Found, 405.22937.

### 1.9—Synthesis of Compound 14

### N-[5-(pyridin-2-yl)pent-4-yn-1-yl]-1,2,3,4-tetrahydroacridin-9-amine (53)

To the degazed solution of 2-bromopyridine (0.093 g, 0.000587 mol, 1 equiv) in CH₃CN/Eu₃N (9 mL/6 mL) catalysts Pd(PPh)₂Cl₂ (0.016 g, 0.00002348 mol, 0.04 equiv) and CuI (0.00447 g, 0.00002348 mol, 0.04 equiv) were added. After degazation and bubbling with Ar and H₂ the mixture was stirred at the room temperature for 5 min, then the degazed solution of alkyne 19 (0.17 g, 0.000645 mol, 1.1 equiv) was added, and the reaction mixture was stirred at 40° C. during 17 h. After concentration at reduced pressure the residue was purified by the flash chromatography (ethyl acetate/MeOH gradient from 100/0 to 90/10) providing 0.14 g of pure compound 53 with 67% yield. R_{f}=0.27 (ethyl acetate/MeOH=9/1). ¹H NMR (300 MHz, CDCl₃, δ): 1.79 (4H, m, CH₂); 2.07 (2H, qui, J=6.7 Hz, CH₂); 2.59 (2H, t, J=6.7 Hz, CH₂); 2.67 (2H, m, CH₂); 3.12 (2H, m, CH₂); 3.92 (2H, t, J=6.7 Hz, CH₂); 5.52 (1H, br s, NH); 7.17-7.21 (1H, m, ArH); 7.27-7.35 (2H, m, ArH); 7.53-7.63 (2H, m, ArH); 8.12 (1H, d, J=8.4 Hz, ArH); 8.19 (1H, d, J=8.4 Hz, ArH); 8.52 (1H, m, ArH). ¹³C NMR (100 MHz, CDCl₃, δ): 17.05; 21.45; 22.36; 24.79; 29.39; 30.63; 47.63; 81.71; 89.07; 113.51; 117.65; 122.77; 123.32; 124.05; 124.72; 126.92; 130.74; 136.26; 141.96; 143.29; 149.85; 153.83.

### N-[5-(pyridin-2-yl)pentyl]-1,2,3,4-tetrahydroacridin-9-amine (51)

To the degazed solution of 53 (0.140 g, 0.00041 mol, 1 equiv) in methanol (2 mL) Pearlman's catalyst Pd(OH)₂/C (20%, moisture 50%, 0.086 g, 0.000123 mol, 0.3 equiv) was added. After the degazation the solution was bubbled with H₂. The reaction mixture was stirred at RT under H₂ at 1 atm for 17 h. The solution was filtered through celite, the solvent was evaporated, and the product was dried in vacuum. The crude product was purified by the column chromatography (CH₂Cl₂/MeOH=100/5) to give 0.14 g of 51 with 56% yield. R_{f}=0.41 (CH₂Cl₂/MeOH=100/5). ¹H NMR (300 MHz, CDCl₃, δ): 1.39-1.49 (2H, m, CH₂); 1.70-1.92 (8H, m, CH₂); 2.61 (2H, J=5.9 Hz, CH₂); 2.75 (2H, J=7.5 Hz, CH₂); 3.21 (2H, J=5.9 Hz, CH₂); 3.88-3.94 (2H, td, J=6.6 Hz, CH₂); 6.49 (1H, t, J=5.5 Hz, NH); 7.03-7.09 (2H, m, ArH); 7.34-7.39 (1H, m, ArH); 7.51-7.61 (2H, m, ArH); 8.20 (1H, d, J=8.7 Hz, ArH); 8.41-8.45 (2H, m, ArH). ¹³C NMR (100 MHz, CDCl₃, δ): 20.79; 22.04; 24.04; 26.28; 28.52; 29.29; 30.89; 37.92; 48.45; 111.05; 115.88; 120.57; 121.35; 123.11; 124.65; 125.32; 132.52; 136.74; 138.83; 149.19; 151.16; 155.82; 161.67.

### 9-(Pent-4-yn-1-ylsulfanyl)-1,2,3,4-tetrahydroacridine (46)

To the solution of compound 44 (2.70 g, 12.54 mmol, 1 equiv) and tetra-n-butylammonium bromide (0.50 g, 1.57 mmol, 0.14 equiv) in 70 mL of THF alkylating agent 45 (3.72 mL, 15.78 mmol, 1.25 equiv) was added followed by 50 mL of aqueous solution of NaOH (50%). The reaction mixture was stirred at reflux during 3 h. The organic phase was separated, the aqueous phase was extracted with ethyl acetate. The organic phases were combined, washed with water, dried under Na₂SO₄ and evaporated. The crude material was purified by flash chromatography to give 1.97 g of 46 (yellow oil, 56% yield). ¹H NMR (400 MHz, CDCl₃, δ): 8.46 (1H, dd, J=8.4, 1.1 Hz), 7.96 (1H, d, J=8.4 Hz), 7.63-7.59 (1H, m), 7.52-7.48 (1H, m), 3.20 (2H, t, J=6.3 Hz), 3.12 (2H, t, J=6.3 Hz), 2.92 (2H, t. J=7.2 Hz), 2.28 (2H, td, J=6.9, 2.7 Hz), 1.98-1.86 (5H, m), 1.69 (2H, qui, J=7.1 Hz). ¹³C NMR (100 MHz, CDCl₃, δ): 159.27, 146.87, 141.43, 136.08, 129.32, 129.08, 128.87, 126.39, 126.08, 83.19, 69.37, 34.83, 34.48, 29.37, 28.90, 23.28, 22.98, 17.77. HRMS (ESI) m/z: calculated for C₁₈H₂₀NS [M+H]⁻ 282.13164. Found [M+H]⁺ 282.13062.

### Methyl 3-hydroxy-6-[5-(1,2,3,4-tetrahydroacridin-9-ylsulfanyl)pent-1-yn-1-yl]pyridine-2-carboxylate (48)

To the degazed solution of 47 (0.75 g, 3.23 mmol, 1 equiv) in 40 mL of THF/Et₃N (1/1) catalysts Pd(PPh₃)₂Cl₂ (0.124 g, 0.177 mmol, 0.05 equiv) and CuI (0.034 g, 0.177 mmol, 0.05 equiv) were added. After degazation the mixture was stirred at the room temperature for 5 min, then 15 mL of degazed solution of alkyne 46 (1.00 g, 3.55 mmol, 1.1 equiv) was added. The reaction mixture was degassed with hydrogen, then stirred during 22 h at 50° C. in Ar/H₂ atmosphere. After concentration at reduced pressure the residue was purified by the flash chromatography (CH₂Cl₂/MeOH) to give 1.26 g of 48 (90% yield). ¹H NMR (400 MHz, CDCl₃, δ): 8.42 (1H, dd, J=8.4, 1.2 Hz), 7.97 (1H, d, J=8.4 Hz), 7.59-7.55 (1H, m), 7.47-7.43 (1H, m), 7.27 (1H, d, J=8.7 Hz), 7.21 (1H, d, J=8.7 Hz), 3.95 (3H, s), 3.16 (2H, t, J=63 Hz), 3.10 (2H, t, J=6.3 Hz), 2.92 (2H, t, J=7.3 Hz), 2.45 (2H, t, J=6.9 Hz), 1.93-1.80 (4H, m), 1.76-1.69 (2H, m). ¹³C NMR (100 MHz, CDCl₃, δ): 169.61, 159.18, 158.00, 146.50, 141.94, 136.14, 135.41, 133.21, 129.89, 129.07, 129.00, 126.64, 126.54, 126.07, 88.54, 80.42, 53.41, 35.08, 34.42, 29.38, 28.74, 23.19, 22.85, 18.71. HRMS (ESI) m/z: calculated for C₂₅H₂₅N₂O₃S [M+H]⁺=433.15858. Found [M+H]⁺=433.15887.

### Methyl 3-hydroxy-6-[5-(1,2,3,4-tetrahydroacridin-9-ylsulfanyl)pentyl]pyridine-2-carboxylate (49)

To the degazed solution of compound 48 (0.62 g, 1.43 mmol, 1 equiv) in 30 mL of ethyl acetate PtO₂ (0.49 g, 2.15 mmol, 1.5 equiv) was added. The reaction mixture was stirred under H₂ at 5 bars during 21 h at rt. The solution was filtered through celite, the solvent was evaporated, and the product was dried in vacuum. The crude product was purified by preparative TLC (c-hexane/ethyl acetate/Et₃N, 3/2/0.05) yielding 0.4 g of 49 (64%). ¹H NMR (400 MHz, CDCl₃, δ): 10.55 (1H, br. s.), 8.41 (1H, d, J=8.2 Hz), 7.94 (1H, d, J=8.2 Hz), 7.58 (1H, t, J=7.6 Hz), 7.46 (1H, t, J=7.6 Hz), 7.22 (1H, d, J=8.7 Hz), 7.16 (1H, d, J=8.7 Hz), 3.97 (3H, s), 3.15 (2H, t, J=6.3 Hz), 3.10 (2H, t, J=6.3 Hz), 2.73 (2H, t, J=7.3 Hz), 2.68 (2H, t, J=7.8 Hz), 1.94-1.82 (4H, m), 1.62-1.47 (4H, m), 1.42-1.34 (2H, m). ¹³C NMR (100 MHz, CDCl₃, δ): 170.23, 159.08, 157.30, 153.84, 146.64, 142.08, 135.85, 129.21, 129.11, 129.01, 128.92, 128.78, 126.75, 126.23, 126.08, 53.28, 37.56, 36.01, 34.36, 30.13, 29.64, 29.28, 28.59, 23.22, 22.90. HRMS (ESI) m/z: calculated for C₂₅H₂₉N₂O₃S [M+H]⁺ 437.18988. Found [M+H]⁺ 437.19191.

### 3-Hydroxy-6-[5-(1,2,3,4-tetrahydroacridin-9-ylsulfanyl)pentyl]pyridine-2-carbaldehyde (50)

Compound 50 was obtained as an orange oil using general procedure 4 (0.088 g, 42% (over three steps). ¹H NMR (400 MHz, CDCl₃, δ): 10.61 (1H, br. s.), 9.97 (1H, s), 8.43 (1H, d, J=8.2 Hz), 7.96 (1H, d, J=8.2 Hz), 7.60 (1H, m), 7.48 (1H, m), 7.23 (1H, d, J=8.7 Hz), 7.18 (1H, d, J=8.7 Hz), 3.17 (2H, t, J=6.3 Hz), 3.12 (2H, t, J=6.5 Hz), 2.78 (2H, t, J=7.3 Hz), 2.69 (2H, t, J=7.7 Hz), 1.97-1.84 (4H, m), 1.67-1.60 (2H, m, J=7.7 Hz), 1.58-1.50 (2H, m, J=7.3 Hz); 1.44-1.38 (2H, m). ¹³C NMR (100 MHz, CDCl₃, δ): 198.89, 159.15, 157.14, 154.78, 146.67, 142.15, 135.91, 129.82, 129.17, 129.06, 128.85, 126.52, 126.30, 126.12, 37.27, 36.05, 34.39, 30.14, 29.33, 29.28, 28.54, 23.27, 22.94. HRMS (ESI) m/z: calculated for C₂₄H₂₇N₂O₂S [M+H]⁺ 407.17932. Found for [M+H]⁺ 407.17692.

### 2-[1-(hydroxyimino)methyl]-6-[5-(1,2,3,4-tetrahydroacridin-9-ylsulfanyl)pentyl]pyridin-3-ol (14)

Compound 14 was obtained accordingly to the general procedure 5 using aldehyde 50 (0.143 g, 0.000352 mol, 1 equiv), NH₂OH.HCl (0.037 g, 0.000528 mol, 1.5 equiv), CH₃COONa (0.036 g, 0.000528 mol, 1.5 equiv), and 5 mL of ethanol. After evaporation of the solvent the crude product was purified by flash chromatography (c-hexane/ethyl acetate) to give compound 14 as a colorless solid (0.085 g, 57%). ¹H NMR (400 MHz, DMSO-d6, δ): 11.81 (1H, s), 10.07 (1H, s), 8.39 (1H, d, J=8.4 Hz), 8.24 (1H, s), 7.89 (1H, d, J=8.4 Hz), 7.68-7.64 (1H, m), 7.58-7.54 (1H, m), 7.23 (1H, d, J=8.5 Hz), 7.05 (1H, d, J=8.5 Hz), 3.12 (2H, t, J=6.2 Hz) 3.02 (2H, t, J=6.3 Hz), 2.82 (2H, t, J=7.0 Hz), 2.56 (2H, t, J=7.5 Hz), 1.90-1.79 (4H, m), 1.56-1.49 (2H, m), 1.47-1.40 (2H, m), 1.38-1.29 (2H, m). ¹³C NMR (100 MHz, DMSO-d6, δ): 158.63, 152.59, 151.35, 151.07, 145.99, 140.62, 135.48, 135.27, 128.80, 128.60, 128.19, 126.20, 125.48, 123.95, 123.66, 36.17, 35.14, 33.55, 29.24, 28.67, 28.46, 27.54, 22.46, 22.09. HRMS (ESI) m/z: calculated for C₂₄H₂₈N₃O₂S [M+H]⁺ 422.19022. Found [M+H]⁺ 422.18876.

### 1.10—Synthesis of Compound 15

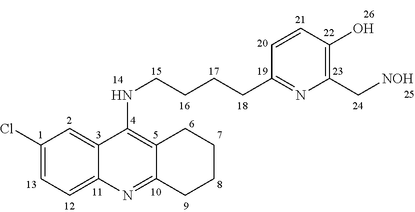

### 7,9-Dichloro-1,2,3,4-tetrahydroacridine

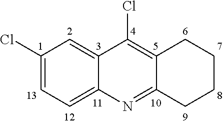 [SMILES: Clc1ccc2nc3c(c(Cl)c2c1)CCCC3]

To a dried flask containing a mixture of commercially available 2-amino-5-benzoic acid (5 g, 29.14 mmol, 1 equiv) and cyclohexanone (3.31 mL, 32.05 mmol, 1.1 equiv), POCl₃ (23.9 mL, 8.8 equiv) was added at 0° C. The mixture was refluxed for 16 h. Upon completion the mixture was poured onto ice and water. The solution was brought to alkalinity using a saturated solution of K₂CO₃. Then chloroform was added and the organic layer was removed and washed with water (200 mL) followed by brine (200 mL). The organic layer was dried over Na₂SO₄, filtered and concentrated under reduced pressure. The crude black oil was purified by column chromatography (petroleum ether/EtOAc 9:1) to afford the desired 7,9-dichloro-1,2,3,4-tetrahydroacridineas sand-coloured solid (7.35 g, 29.14 mmol, quant). Spectroscopic data were consistent with those reported in literature [Cross, R. M.; Maignan, J. R.; Mutka, T. S.; Luong, L.; Sargent, J.; Kyle, D. E.; Manetsch, R. Optimization of 1,2,3,4-Tetrahydroacridin-9(10H)-ones as Antimalarials Utilizing Structure-Activity and Structure-Property Relationships. J. Med. Chem. 2011, 9, 4399-4426].

### 7-Chloro-1,2,3,4-tetrahydroacridin-9-(10H)-one

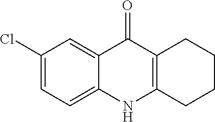 [SMILES: O=c1c2c([nH]c3ccc(Cl)cc13)CCCC2]

7,9-Dichloro-1,2,3,4-tetrahydroacridine (2.00 g, 7.93 mmol) was dissolved in glacial acetic acid (20 mL, 0.4 M) and the reaction mixture was heated in a sealed tube at 200° C. for 24 h. The crude material was poured onto ice and water. The precipitate was filtered, co-evaporated with toluene and the powder was dried in vacuo afforded the desired 7-chloro-1,2,3,4-tetrahydroacridin-9-(10H)-one as a sand-coloured solid (1.85 g, 7.93 mmol, quant.) Spectroscopic data were consistent with those reported in literature [Cross, R. M.; Maignan, J. R.; Mutka, T. S.; Luong, L.; Sargent, J.; Kyle, D. E.; Manetsch, R. Optimization of 1,2,3,4-Tetrahydroacridin-9(10H)-ones as Antimalarials Utilizing Structure-Activity and Structure-Property Relationships. J. Med. Chem. 2011, 9, 4399-4426].

### 7-Chloro-1,2,3,4-tetrahydroacridin-9-yl trifluoromethanesulfonate

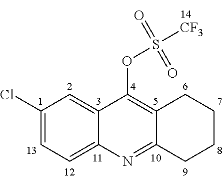 [SMILES: O=S(=O)(Oc1c2c(nc3ccc(Cl)cc13)CCCC2)C(F)(F)F]

To a suspension of dried triethylamine (1.15 mL, 8.28 mmol, 1.1 equiv) and 7-chloro-1,2,3,4-tetrahydroacridin-9-(10H)-one (1.76 g, 7.53 mmol, 1 equiv) in freshly distilled CH₂Cl₂ (80 mL, 0.1 M), triflic anhydride (1.39 mL, 8.28 mmol, 1.1 equiv) was added dropwise at −78° C. The reaction mixture was stirred 1 h at −78° C. and was then allowed to warm up at room temperature while stirring was continued for 16 h. Upon completion, the solvent was removed under reduced pressure and the remaining dark yellow oil was purified by column chromatography (petroleum ether/EtOAc 4:1) to afford the desired 7-chloro-1,2,3,4-tetrahydroacridin-9-yl trifluoromethanesulfonate as white solid (2.48 g, 6.78 mmol, 90%).

TLC R_{f} (petroleum ether/EtOAc 4:1) 0.40.

MP 86-87° C.

IR νₘₐₓ neat (cm⁻¹) 2945, 2869, 1601, 1477, 1412, 1344, 1323, 1212, 1134, 1082, 1018.

¹H NMR (400 MHz, CDCl₃) δ (ppm) 7.96 (d, J=9.0 Hz, 1H, 12), 7.93 (d, J=2.2 Hz, 1H, 2), 7.64 (dd, J=2.3, 9.1 Hz, 1H, 13), 3.16 (t, J=6.6 Hz, 2H, 9), 3.04 (t, J=6.5 Hz, 2H, 6), 2.04-1.89 (m, 4H, 7+8).

¹³C NMR (101 MHz, CDCl₃) δ (ppm) 161.57 (10), 148.82 (4), 146.46 (11), 133.57 (1), 131.13 (13), 130.48 (12), 125.33 (3), 121.60 (5), 119.93 (2), 118.79 (q, J 316.4 Hz, 14), 33.79 (9), 24.32 (6), 22.42 (8), 21.93 (7).

¹⁹F-NMR (376 MHz, CDCl₃) δ (ppm) −72.64.

LRMS (ESI⁺) m/z 366.0 ([M+H]⁺).

HRMS (ESI⁺) m/z calcd for C₁₄H₁₂³⁵ClF₃NO₃S⁺ 366.0182 found 366.0183 Da.

### N-(But-3-yn-1-yl)-7-chloro-1,2,3,4-tetrahydroacridin-9-amine

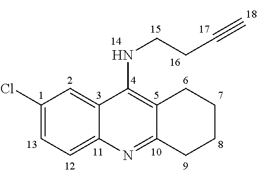 [SMILES: C#CCCNc1c2c(nc3ccc(Cl)cc13)CCCC2]

A microwave tube containing a magnetic stirrer bar was charged with 7-chloro-1,2,3,4-tetrahydroacridin-9-yl trifluoromethanesulfonate (100 mg, 0.273 mmol, 1 equiv), Pd₂(dba)₃ (10 mg, 4 mol %), (±)-BINAP (14 mg, 8 mol %) and Cs₂CO₃ (222 mg, 0.683 mmol, 2.5 equiv). The vessel was sealed with a microwave septum and purged with argon. Degassed 1,4-dioxane (3 mL, 9.10⁻² M) and 1-amino-3-butyne (29 μL, 0.355 mmol, 1.3 equiv) were introduced through the septum. The resulting mixture was heated to 100° C. for 1 husing a Biotage® Initiator Microwave Synthesizer Apparatus. After cooling, the reaction mixture was concentrated and purified by column chromatography (Et₂O 100%) to afford the desired N-(but-3-yn-1-yl)-7-chloro-1,2,3,4-tetrahydroacridin-9-amine as yellow oil (66 mg, 85%).

TLC R_{f} (Et₂O 100%) 0.23.

IR νₘₐₓ neat (cm⁻¹) 3301, 2934, 2861, 1581, 1555, 1487, 1433, 1349, 1338, 1126.

¹H NMR (400 MHz, CDCl₃) δ (ppm) 7.95 (d, J=2.3 Hz, 1H, 2), 7.83 (d, J=9.0 Hz, 1H, 12), 7.48 (dd, J=2.2, 8.9 Hz, 1H, 13), 4.23 (t, J=6.9 Hz, 1H, 14), 3.57 (q, J=6.4 Hz, 2H, 15), 3.04 (t, J=6.0 Hz, 2H, 9), 2.79 (t, J=6.4 Hz, 2H, 6), 2.46 (td, J=2.7, 6.2 Hz, 2H, 16), 2.14 (t, J=2.6 Hz, 1H, 18), 1.96-1.90 (m, 4H, 7+8).

¹³C NMR (101 MHz, CDCl₃) δ (ppm) 159.34 (10), 149.23 (4), 146.00 (11), 130.71 (13), 129.79 (12), 129.29 (1), 121.97 (2), 121.75 (3), 118.94 (5), 81.65 (17), 71.17 (18), 47.56 (15), 34.21 (9), 24.87 (6), 23.05 (8), 22.85 (7), 21.03 (16).

LRMS (ESI⁺) m/z 285.1 ([M+H]⁺).

HRMS (ESI⁺) m/z calcd for C₇H₁₈³⁵ClN₂⁺ 285.1153 found 285.1159 Da.

### Methyl 3-(benzyloxy)-6-(4-((7-chloro-1,2,3,4-tetrahydroacridin-9-yl)amino)but-1-yn-1-yl)picolinate

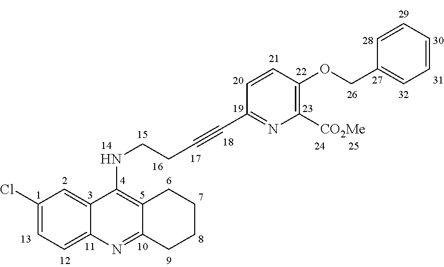 [SMILES: COC(=O)c1nc(C#CCCNc2c3c(nc4ccc(Cl)cc24)CCCC3)ccc1OCc1ccccc1]

To a degassed solution of methyl 3-benzyloxy-6-bromopicolinate (124 mg, 0.386 mmol, 1.1 equiv) in THF/Et₃N (3 mL/2 mL), Pd[PPh₃]₄ (40 mg, 0.035 mmol, 0.1 equiv) and CuI (13 mg, 0.070 mmol, 0.2 equiv) were added. After degassing the reaction mixture 5 min at room temperature, a degassed solution of N-(but-3-yn-1-yl)-7-chloro-1,2,3,4-tetrahydroacridin-9-amine (100 mg, 0.351 mmol, 1 equiv) in THF (1 mL) was added and the reaction mixture was stirred at the room temperature for 16 h. After completion, the reaction mixture was concentrated under reduced pressure and the residue was purified by column chromatography (EtOAc 100%) to afford the desired methyl 3-(benzyloxy)-6-(4-((7-chloro-1,2,3,4-tetrahydroacridin-9-yl)amino)but-1-yn-1-yl)picolinateas yellow oil (157 mg, 0.298 mmol, 85%).

TLC R_{f} (EtOAc 100%) 0.23.

IR νₘₐₓ neat (cm⁻¹) 3368, 3062, 2932, 2862, 2232, 1733, 1580, 1557, 1486, 1450, 1435, 1382, 1349, 1290, 1270, 1209, 1098.

¹H NMR (400 MHz, CDCl₃) δ (ppm) 7.96 (d, J=2.4 Hz, 1H, 2), 7.86 (d, J=8.9 Hz, 1H, 12), 7.48-7.27 (m, 8H, 13+20+21+28+29+30+31+32), 5.21 (s, 2H, 26), 4.31 (t, J=6.6 Hz, 1H, 14), 3.96 (s, 3H, 25), 3.68 (q, J=6.4 Hz, 2H, 15), 3.03 (t, J=5.9 Hz, 2H, 9), 2.80 (t, J=6.0 Hz, 2H, 6), 2.68 (t, J=6.2 Hz, 2H, 16), 1.87-1.85 (m, 4H, 7+8).

¹³C NMR (101 MHz, CDCl₃) δ (ppm) 164.88 (24), 159.25 (10), 153.33 (22), 149.31 (4), 145.80 (11), 140.43 (23), 135.51 (19), 134.65 (27), 130.57 (13), 130.10 (20), 129.81 (1), 129.31 (12), 128.89 (29+31), 128.42 (30), 127.04 (28+32), 121.88 (2), 121.86 (21), 121.62 (3), 118.84 (5), 86.68 (17), 81.80 (18), 70.98 (26), 52.87 (25), 47.36 (15), 34.15 (9), 25.03 (6), 22.96 (7), 22.73 (8), 22.01 (16).

LRMS (ESI⁺) m/z 526.2 ([M+H]⁺).

HRMS (ESI⁺) m/z calcd for C₃₁H₂₉³⁵ClN₃O₃⁺ 526.1897 found 526.1895 Da.

### Methyl 6-(4-((7-chloro-1,2,3,4-tetrahydroacridin-9-yl)amino)butyl)-3-hydroxypicolinate

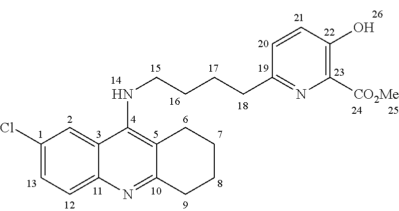 [SMILES: COC(=O)c1nc(CCCCNc2c3c(nc4ccc(Cl)cc24)CCCC3)ccc1O]

To a degassed solution of methyl 3-(benzyloxy)-6-(4-((7-chloro-1,2,3,4-tetrahydroacridin-9-yl)amino)but-1-yn-1-yl)picolinate (97 mg, 0.184 mmol) in EtOAc (3 mL, 0.06 M), Pearlman's catalyst Pd(OH)₂/C (20% with 50% moisture, 51 mg, 0.04 mmol, 0.2 equiv) was added. After evaporating and flushing with Hz five times, the reaction mixture was stirred 7 h at room temperature under H₂ (1 atm.). Upon completion, the catalyst was removed by filtration through a short column of celite, the solvent was evaporated to afford the desired methyl 6-(4-((7-chloro-1,2,3,4-tetrahydroacridin-9-yl)amino)butyl)-3-hydroxypicolinate as pale yellow oil (65 mg, 0.147 mmol, 80%).

TLC R_{f} (CH₂Cl₂/MeOH 9:1) 0.4.

IR νₘₐₓ neat (cm⁻¹) 3450, 2925, 2855, 1674, 1579, 1467, 1445, 1354, 1296, 1212, 1101.

¹H NMR (500 MHz, CDCl₃) δ (ppm) 10.60 (br s, 1H, 26), 7.92 (d, J=2.2 Hz, 1H, 2), 7.86 (d, J=9.1 Hz, 1H, 12), 7.48 (dd, J=2.3, 9.0 Hz, 1H, 13), 7.30 (d, J=8.8, 1H, 21), 7.25 (d, J=8.7, 1H, 20), 4.02 (s, 3H, 25), 3.50 (t, J=7.0 Hz, 2H, 15), 3.04 (m, 2H, 9), 2.83 (t, J=7.5 Hz, 2H, 18), 2.66 (m, 2H, 6), 2.16 (s, 1H, 14), 1.92-1.87 (m, 4H, 7+8), 1.86-1.79 (m, 2H, 17), 1.78-1.68 (m, 2H, 16).

¹³C NMR (126 MHz, CDCl₃) δ (ppm) 170.17 (24), 158.54 (10), 157.43 (19), 153.28 (22), 150.26 (4), 145.11 (11), 130.17 (23), 129.41 (12), 129.28 (20), 129.04 (13), 128.81 (1), 126.93 (21), 122.15 (2), 120.84 (3), 116.75 (5), 53.31 (25), 49.30 (15), 37.16 (18), 33.82 (9), 31.29 (16), 27.25 (17), 24.87 (6), 22.99 (7), 22.68 (8).

LRMS (ESI⁺) m/z 440.2 ([M+H]⁺).

HRMS (ESI⁺) m/z calcd for C₂₄H₂₇³⁵ClN₃O⁺ 440.1741 found 440.1745 Da.

### 6-(4-((7-Chloro-1,2,3,4-tetrahydroacridin-9-yl)amino)butyl)-3-hydroxypicolinaldehyde oxime

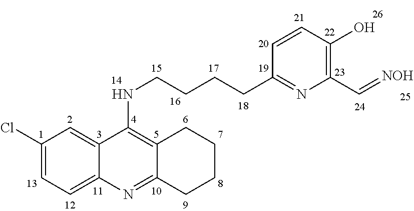

A solution of methyl 6-(4-((7-chloro-1,2,3,4-tetrahydroacridin-9-yl)amino)butyl)-3-hydroxypicolinate (50 mg, 0.113 mmol, 1 equiv), imidazole (31 mg, 0.455 mmol, 4 equiv), and tert-butyldimethylsilyl chloride (38 mg, 0.251 mmol, 2.2 equiv) in dry DMF (1.1 mL, 0.1 M) was stirred at the room temperature under argon atmosphere during 2 h. After completion, ethyl acetate was added, and the organic phase was washed with water thrice, dried over Na₂SO₄ and concentrated under reduced pressure. LRMS (ESI⁺) m/z 554.2 [M+H]⁺.

After drying in vacuo, the residue was subjected to the following step. To the solution of methyl 3-((tert-butyldimethylsilyl)oxy)-6-(4-((7-chloro-1,2,3,4-tetrahydroacridin-9-yl)amino)butyl)picolinate (40 mg, 0.072 mmol, 1 equiv) in dry CH₂Cl₂ (1.8 mL, 0.04 M) at −78° C., DIBAL-H (1M solution in CH₂Cl₂, 144 μL, 0.144 mmol, 2 equiv) was added dropwise. The reaction mixture was stirred at −78° C. for 12 min, then the reaction was quenched with MeOH (2 mL), and the cooling bath was removed. When the mixture was warmed to room temperature the organic phase was washed with aqueous solution of NaOH (1M), dried over Na₂SO₄ and concentrated under reduced pressure. LRMS (ESI⁺) m/z 410.0 [M-TBS+H]⁺.

After drying in vacuo, the residue was subjected to the following step. To the solution of 3-((tert-butyldimethylsilyl)oxy)-6-(4-((7-chloro-1,2,3,4-tetrahydroacridin-9-yl)amino)butyl)picolinaldehyde (40 mg, 0.076 mmol, 1 equiv) in dry THF (1.4 mL, 0.05 M) at 0° C., TBAF (1M solution in THF, 84 μL, 0.084 mmol, 1.1 equiv) was added, and the reaction mixture was stirred at 0° C. for 1 h. After completion, the solvent was removed under reduced pressure. LRMS (ESI⁺) m/z 410.0 [M+H]⁺.

After drying in vacuo, the residue was subjected to the following step. A solution of 6-(4-((7-chloro-1,2,3,4-tetrahydroacridin-9-yl)amino)butyl)-3-hydroxypicolinaldehyde (20 mg, 0.049 mmol, 1 equiv), hydroxylamine hydrochloride (5 mg, 0.073 mmol, 1.5 equiv), and anhydrous CH₃CO₂Na (6 mg, 0.073 mmol, 1.5 equiv) in dry ethanol (1 mL, 0.05 M) was stirred at reflux during 15 h. After concentration under reduced pressure, the crude material was purified by the column chromatography (CH₂Cl₂/MeOH 9:1) to afford 6-(4-((7-Chloro-1,2,3,4-tetrahydroacridin-9-yl)amino)butyl)-3-hydroxypicolinaldehyde oxime as yellow solid (7 mg, 0.016 mmol, 15% over 4 steps). TLC R_{f} (CH₂Cl₂/MeOH 9:1) 0.23.

IR νₘₐₓ neat (cm⁻¹) 2927, 2857, 2760, 1576, 1561, 1492, 1465, 1270, 1167, 1028.

¹H NMR (500 MHz, CDCl₃) δ (ppm) 10.16 (br s, 2H, 26+25), 8.25 (s, 1H, 24), 7.87-7.85 (m, 2H, 2+12), 7.48 (dd, J=1.8, 9.1 Hz, 1H, 13), 7.06 (d, J=8.52 Hz, 1H, 20), 6.92 (d, J=8.37 Hz, 1H, 21), 4.08 (br s, 1H, 14), 3.52 (t, J=6.7 Hz, 2H, 15), 3.06 (m, 2H, 9), 2.75 (t, J=7.1 Hz, 2H, 18), 2.61 (m, 2H, 6), 1.94-1.88 (m, 4H, 7+8), 1.87-1.83 (m, 2H, 17), 1.76-1.72 (m, 2H, 16).

¹³C NMR (126 MHz, CDCl₃) δ (ppm) 158.04 (24), 153.12 (19), 152.59 (10), 152.45 (22), 150.56 (23), 144.86 (11), 135.55 (24), 129.71 (21), 129.29 (20), 129.22 (1), 124.34 (13), 123.73 (21), 122.28 (2), 120.13 (3), 115.75 (5), 48.81 (15), 36.73 (18), 33.05 (9), 30.99 (16), 26.56 (17), 24.69 (6), 22.85 (7), 22.47 (8).

LRMS (ESI⁺) m/z 425.2 ([M+H]⁺).

HRMS (ESI⁺) m/z calcd for C₂₃H₂₆³⁵ClN₄O₂⁺ 425.1744 found 425.1746 Da.

### 1.11—Synthesis of Compound 16

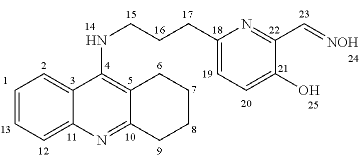

### 1,2,3,4-tetrahydroacridin-9-yl trifluoromethanesulfonate

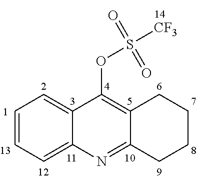 [SMILES: O=S(=O)(Oc1c2c(nc3ccccc13)CCCC2)C(F)(F)F]

To a suspension of dried triethylamine (1.53 mL, 11.04 mmol, 1.1 equiv) and 1,2,3,4-tetrahydroacridin-9-(10H)-one (2 g, 10.04 mmol, 1 equiv) in freshly distilled CH₂Cl₂ (100 mL, 0.1 M), triflic anhydride (1.86 mL, 11.04 mmol, 1.1 equiv) was added dropwise at −78° C. The reaction mixture was stirred 1 h at −78° C. and was then allowed to warm up at room temperature while stirring was continued for 16 h. Upon completion, the solvent was removed under reduced pressure and the remaining dark yellow oil was purified by column chromatography (hexane/EtOAc 4:1) to afford the desired 1,2,3,4-tetrahydroacridin-9-yl trifluoromethanesulfonate as white solid (2.83 g, 8.53 mmol, 85%).

TLC R_{f} (petroleum ether/EtOAc 1:1) 0.80.

MP 55-56° C.

IR νₘₐₓ neat (cm⁻¹) 2946, 2875, 1626, 1603, 1556, 1487, 1405, 1326, 1201, 1128, 1003.

¹H NMR (400 MHz, CDCl₃) δ (ppm) 8.04 (d, J=8.6 Hz, 1H, 2), 7.99 (d, J=8.4 Hz, 1H, 12), 7.72 (t, J=8.0 Hz, 1H, 1), 7.59 (t, J=7.7 Hz, 1H, 13), 3.18 (t, J=6.6 Hz, 2H, 9), 3.05 (t, J=6.4 Hz, 2H, 6), 2.04-1.88 (m, 4H, 7+8).

¹³C NMR (101 MHz, CDCl₃) δ (ppm) 161.12 (10), 149.99 (4), 148.10 (11), 130.09 (13), 128.77 (12), 127.42 (1), 124.14 (3), 120.87 (5), 120.82 (2), 118.78 (q, J=319.9 Hz, 14), 33.83 (9), 24.24 (6), 22.53 (8), 22.04 (7).

¹⁹F NMR (376 MHz, CDCl₃) δ (ppm) −72.87.

LRMS (ESI⁺) m/z 332.1 ([M+H]⁺).

HRMS (ESI⁺) m/z calcd for C₄₁H₁₃F₃NO₃S⁺ 332.0563 found 332.0563 Da.

### N-(prop-2-yn-1-yl)-1,2,3,4-tetrahydroacridin-9-amine

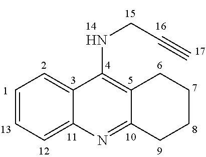 [SMILES: C#CCNc1c2c(nc3ccccc13)CCCC2]

A microwave tube containing a magnetic stirrer bar was charged with 1,2,3,4-tetrahydroacridin-9-yl trifluoromethanesulfonate (100 mg, 0.302 mmol, 1 equiv), Pd₂(dba)₃ (11 mg, 4 mol %), (±)-BINAP (16 mg, 8 mol %) and Cs₂CO₃ (246 mg, 0.755 mmol, 2.5 equiv). The vessel was sealed with a microwave septum and purged with argon. Degassed 1,4-dioxane (3 mL, 9.10⁻² M) and propargylamine (25 μL, 0.392 mmol, 1.3 equiv) were introduced through the septum. The resulting mixture was heated to 100° C. for 1 husing a Biotage® Initiator Microwave Synthesizer Apparatus. After cooling, the reaction mixture was concentrated and purified by column chromatography (Et₂O/MeOH 9:1) to afford the desired N-(prop-2-yn-1-yl)-1,2,3,4-tetrahydroacridin-9-amine as yellow solid (64 mg, 0.272 mmol, 90%).

MP 120° C.

TLC R_{f} (Et₂O/MeOH 9:1) 0.3.

IR νₘₐₓ neat (cm⁻¹) 3291, 3063, 2933, 2861, 2113, 1615, 1582, 1562, 1497, 1434, 1407, 1375, 1330, 1168, 1111.

¹H NMR (400 MHz, CDCl₃) δ (ppm) 7.93 (t, J=7.8 Hz, 2H, 2+12), 7.58 (t, J=7.3 Hz, 1H, 13), 7.41 (t, J=7.7 Hz, 1H, 1), 4.17 (br s, 3H, 14+15), 3.10 (t, J=5.9 Hz, 2H, 9), 2.85 (t, J=5.8 Hz, 2H, 6), 2.28 (s, 1H, 17), 1.98-1.87 (m, 4H, 7+8).

¹³C NMR (101 MHz, CDCl₃) δ (ppm) 159.10 (10), 149.44 (4), 147.51 (11), 129.11 (13), 128.63 (12), 124.65 (1), 122.48 (2), 121.09 (3), 119.06 (5), 81.35 (16), 72.70 (17), 38.64 (15), 34.23 (9), 24.92 (6), 23.12 (8), 22.91 (7).

LRMS (ESI⁺) m/z 237.0 ([M+H]⁺).

HRMS (ESI⁺) m/z calcd for C₁₆H₁₇N₂⁺ 237.1386 found 237.1383 Da.

### Methyl 3-(benzyloxy)-6-(3-((1,2,3,4-tetrahydroacridin-9-yl)amino)prop-1-yn-1-yl)picolinate

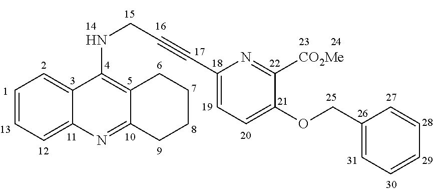 [SMILES: COC(=O)c1nc(C#CCNc2c3c(nc4ccccc24)CCCC3)ccc1OCc1ccccc1]

To a degassed solution of methyl 3-benzyloxy-6-bromopicolinate (225 mg, 0.698 mmol, 1.1 equiv) in THF/Et₃N (4 mL 3 mL), Pd[PPh₃]₄ (73 mg, 0.064 mmol, 0.1 equiv) and CuI (24 mg, 0.128 mmol, 0.2 equiv) were added. After degassing the reaction mixture 5 min at room temperature, a degassed solution of N-(prop-2-yn-1-yl)-1,2,3,4-tetrahydroacridin-9-amine (150 mg, 0.635 mmol, 1 equiv) in THF (2 mL) was added and the reaction mixture was stirred at the room temperature for 16 h. After completion, the reaction mixture was concentrated under reduced pressure and the residue was purified by column chromatography (EtOAc/MeOH from 98:2 to 9:1) to afford the desired methyl 3-(benzyloxy)-6-(3-((1,2,3,4-tetrahydroacridin-9-yl)amino)prop-11-yn-1-yl)picolinate as yellow wax (285 mg, 0.597 mmol, 94%).

TLC R_{f} (EtOAc/MeOH 9:1) 0.23.

IR νₘₐₓ neat (cm⁻¹) 3327, 3063, 2935, 2870, 2089, 1730, 1580, 1562, 1496, 1450, 1434, 1379, 1293, 1267, 1209, 1097.

¹H NMR (400 MHz, CDCl₃) δ (ppm) 7.94 (t, J=7.2 Hz, 2H, 2+12), 7.55 (t, J=7.2 Hz, 1H, 13), 7.42-7.27 (m, 6H, 1+27+28+29+30+31), 7.23-7.17 (m, 2H, 19+20), 5.14 (s, 2H, 25), 4.33 (d, J=5.3 Hz, 2H, 15), 4.25-4.22 (m, 1H, 14), 3.92 (s, 3H, 24), 3.06 (m, 2H, 9), 2.83 (m, 2H, 6), 1.87 (m, 4H, 7+8).

¹³C NMR (101 MHz, CDCl₃) δ (ppm) 164.70 (23), 159.05 (10), 153.36 (21), 149.31 (4), 147.42 (11), 140.37 (22), 135.35 (18), 134.12 (26), 130.12 (19), 128.94 (13), 128.78 (2C, 28+30), 128.51 (12), 128.32 (29), 126.92 (2C, 27+31), 124.56 (1), 122.52 (2), 121.64 (20), 121.31 (3), 119.31 (5), 86.32 (16), 82.93 (17), 70.85 (25), 52.75 (24), 39.23 (15), 34.16 (9), 24.89 (6), 22.97 (8), 22.79 (7).

LRMS (ESI⁺) m/z 478.0 ([M+H]⁺).

HRMS (ESI⁺) m/calcd for C₃₀H₂₅N₃O₃⁺ 478.2125 found 478.2127 Da.

### Methyl 3-hydroxy-6-(3-((1,2,3,4-tetrahydroacridin-9-yl)amino)propyl)picolinate

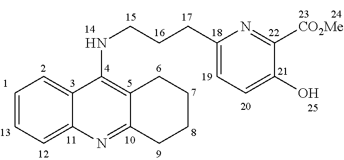 [SMILES: COC(=O)c1nc(CCCNc2c3c(nc4ccccc24)CCCC3)ccc1O]

To a degassed solution of methyl 3-(benzyloxy)-6-(3-((1,2,3,4-tetrahydroacridin-9-yl)amino)prop-1-yn-1-yl)picolinate (100 mg, 0.209 mmol, 1 equiv) in dry MeOH (2.1 mL, 0.1 M), Pearlman's catalyst Pd(OH)₂/C (20% with 50% moisture, 88 mg, 0.06 mmol, 0.3 equiv) was added. After evaporating and flushing with H₂ five times, the reaction mixture was stirred 2 h at room temperature under H₂ (1 atm.). Upon completion, the catalyst was removed by filtration through a short column of celite, the solvent was evaporated and the residue was purified by column chromatography (CH₂Cl₂/MeOH 9:1) to afford the desired methyl 3-hydroxy-6-(3-((1,2,3,4-tetrahydroacridin-9-yl)amino)propyl)picolinateas white solid (79 mg, 0.203 mmol, 97%).

TLC R_{f} (CH₂Cl₂/MeOH 9:1) 0.40.

MP 108° C.

IR νₘₐₓ neat (cm⁻¹) 3243, 3044, 2923, 2845, 1728, 1672, 1635, 1575, 1523, 1466, 1445, 1363, 1329, 1307, 1211, 1180, 1102.

¹H NMR (400 MHz, CDCl₃) δ (ppm) 10.54 (br s, 1H, 25), 8.48 (t, J=6.2 Hz, 1H, 12), 8.24 (d, J=8.7 Hz, 1H, 2), 7.64 (t, J=7.5 Hz, 1H, 13), 7.43-7.28 (m, 3H, 1+19+20), 6.82 (br s, 1H, 14), 4.03-3.92 (m, 5H, 15+24), 3.28 (m, 2H, 9), 2.92 (t, J=7.0 Hz, 2H, 17), 2.77-2.55 (m, 2H, 6), 2.22 (quin, J=6.5 Hz, 2H, 16), 1.95-1.76 (m, 4H, 7+8).

¹³C NMR (101 MHz, CDCl₃) δ (ppm) 169.80 (23), 157.57 (18), 155.46 (10), 151.97 (21), 139.93 (4), 139.28 (11), 132.07 (13), 129.75 (19), 128.94 (22), 127.40 (12), 125.20 (20), 123.93 (1), 121.49 (2), 116.49 (3), 111.35 (5), 53.26 (24), 46.78 (16), 33.80 (17), 30.21 (9), 28.91 (16), 24.46 (6), 22.25 (7), 20.90 (8).

LRMS (ESI⁺) m/z 391.9 ([M+H]⁺).

HRMS (ESI⁺) m/z calcd for C₂₃H₂₆N₃O₃⁺ 392.1969 found 392.1969 Da.

### 3-Hydroxy-6-(3-((1,2,3,4-tetrahydroacridin-9-yl)amino)propyl)picolinaldehyde oxime

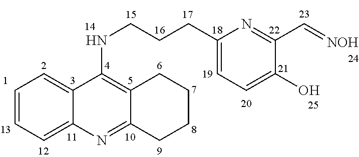

A solution of methyl 3-hydroxy-6-(3-((1,2,3,4-tetrahydroacridin-9-yl)amino)propyl)picolinate (14 mg, 0.037 mmol, 1 equiv), imidazole (10 mg, 0.148 mmol, 4 equiv), and tert-butyldimethylsilyl chloride (12 mg, 0.08 mmol, 2.2 equiv) in dry DMF (1 mL, 0.04 M) was stirred at the room temperature under argon atmosphere during 2 h. After completion, ethyl acetate was added, and the organic phase was washed with water thrice, dried over Na₂SO₄ and concentrated under reduced pressure.

LRMS (ESI⁺) m/z 506.1 [M+H]⁺.

After drying in vacuo, the residue was subjected to the following step. To the solution of methyl 3-((tert-butyldimethylsilyl)oxy)-6-(3-((1,2,3,4-tetrahydroacridin-9-yl)amino)propyl)picolinate (13 mg, 0.026 mmol, 1 equiv) in dry CH₂Cl₂ (1 mL, 0.03 M) at −78° C., DIBAL-H (1M solution in CH₂Cl₂, 52 μL, 0.052 mmol, 2 equiv) was added dropwise. The reaction mixture was stirred at −78° C. for 12 min, then the reaction was quenched with MeOH (2 mL), and the cooling bath was removed. When the mixture was warmed to room temperature the organic phase was washed with aqueous solution of NaOH (1M), dried over Na₂SO₄ and concentrated under reduced pressure. LRMS (ESI⁺) m/z 476.2 [M+H]⁺.

After drying in vacuo, the residue was subjected to the following step. To the solution of 3-((tert-butyldimethylsilyl)oxy)-6-(3-((1,2,3,4-tetrahydroacridin-9-yl)amino)propyl) picolinaldehyde (6 mg, 0.013 mmol, 1 equiv) in dry THF (1 mL, 0.01 M) at 0° C., TBAF (1M solution in THF, 14 μL, 0.014 mmol, 1.1 equiv) was added, and the reaction mixture was stirred at 0° C. for 1 h. After completion, the solvent was removed under reduced pressure. LRMS (ESI⁺) m/z 362.0 [M+H]⁺ (4 mg, 0.011 mmol, 30% over 3 steps).

After drying in vacuo, the residue was subjected to the following step. A solution of 3-hydroxy-6-(3-((1,2,3,4-tetrahydroacridin-9-yl)amino)propyl)picolinaldehyde (4 mg, 0.012 mmol, 1 equiv), hydroxylamine hydrochloride (1.25 mg, 0.018 mmol, 1.5 equiv), and anhydrous CH₃CO₂Na (1.5 mg, 0.018 mmol, 1.5 equiv) in dry ethanol (1 mL, 0.01 M) was stirred at reflux during 15 h. After concentration under reduced pressure, the crude material was purified by the column chromatography (CH₂Cl₂/MeOH 9:1) to afford 3-Hydroxy-6-(3-((1,2,3,4-tetrahydroacridin-9-yl)amino)propyl)picolinaldehyde oxime as yellow solid (4 mg, 0.011 mmol, 86%).

TLC R_{f} (CH₂Cl₂/MeOH 9:1) 0.21.

IR νₘₐₓ neat (cm⁻¹) 3355, 2927, 2855, 1634, 1575, 1521, 1464, 1416, 1359, 1329, 1272, 1168, 1019.

¹H NMR (400 MHz, CD₃OD) δ (ppm) 8.28 (d, J=8.4 Hz, 1H, 12), 7.97 (s, 1H, 23), 7.84-7.80 (m, 1H, 1), 7.72 (d, J=8.3 Hz, 1H, 2), 7.51 (t, J=7.7 Hz, 3H, 13), 7.19 (d, J=8.5 Hz, 1H, 20), 7.11 (d, J=8.5 Hz, 1H, 19), 4.03 (t, J=6.6 Hz, 2H, 15), 3.01-2.93 (m, 2H, 9), 2.85 (t, J=6.9 Hz, 2H, 17), 2.55-2.53 (m, 2H, 6), 2.25 (quin, J=6.7 Hz, 2H, 16), 1.98 (br s, 1H, 14), 1.96-1.87 (m, 4H, 7+8).

¹³C NMR (101 MHz, CD₃OD) δ (ppm) 157.98 (10), 153.75 (18), 153.11 (21), 152.65 (23), 151.41 (4), 139.72 (11), 136.41 (18), 134.01 (13), 126.49 (19), 126.17 (20), 125.97 (12), 125.47 (1), 120.02 (2), 116.98 (3), 112.74 (5), 48.38 (15), 34.97 (17), 30.68 (9), 29.20 (6), 24.84 (16), 22.88 (7), 21.75 (8).

LRMS (ESI⁺) m/z 377.1 ([M+H]⁺).

HRMS (ESI⁺) m/z calcd for C₂₂H₂₅N₄O₂⁺ 377.1972 found 377.1970 Da.

### 1.12—Synthesis of Compound 17

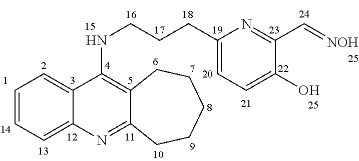

### 2,11-Dichloro-7,8,9,10-tetrahydro-6H-cyclohepta[b]quinoline

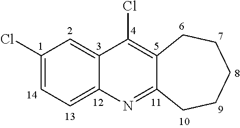 [SMILES: Clc1ccc2nc3c(c(Cl)c2c1)CCCCC3]

To dried flask containing a mixture of commercially available 2-amino-5-benzoic acid (5 g, 29.14 mmol, 1 equiv) and cycloheptanone (2.78 mL, 32.05 mmol, 1.1 equiv), POCl₃ (23.8 mL, 8.8 equiv) was added at 0° C. The mixture was refluxed for 16 h. Upon completion the mixture was poured onto ice and water. The solution was brought to alkalinity using a saturated solution of K₂CO₃. Then chloroform was added and the organic layer was removed and washed with water (200 mL) followed by brine (200 mL). The organic layer was dried over Na₂SO₄, filtered and concentrated under reduced pressure. The crude black oil was purified by column chromatography (petroleum ether/EtOAc 9:1) to afford the desired 2,11-dichloro-7,8,9,10-tetrahydro-6H-cyclohepta[b]quinoline as yellow powder (6.17 g, 23.31 mmol, 80%).

TLC R_{f} (petroleum ether/EtOAc 9:1) 0.31.

IR νₘₐₓ neat (cm⁻¹) 2924, 2853, 1584, 1551, 1475, 1455, 1440, 1335, 1316, 1186, 1080.

¹H NMR (400 MHz, CDCl₃) δ (ppm) 8.13 (d, J=2.3 Hz, 1H, 2), 7.89 (d, J=8.9 Hz, 1H, 13), 7.57 (dd, J=2.3, 8.9 Hz, 1H, 14), 3.22-3.17 (m, 4H, 6+10), 1.92-1.86 (m, 2H, 9), 1.81-1.71 (m, 4H, 7+8).

¹³C NMR (101 MHz, CDCl₃) δ (ppm) 165.27 (11), 144.95 (12), 138.68 (4), 134.99 (5), 132.76 (1), 130.65 (14), 130.10 (13), 126.36 (3), 123.69 (2), 40.33 (10), 31.89 (8), 30.57 (6), 27.50 (9), 26.91 (7).

LRMS (ESI⁺) m/z 266.0 ([M+H]⁺).

HRMS (ESI⁺) m/z calcd for C₁₄H₁₄³⁵Cl₂N⁺ 266.0503. Found 266.0497 Da.

### 2-Chloro-5,6,7,8,9,10-hexahydro-11H-cyclohepta[b]quinolin-11-one

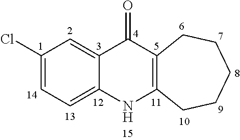 [SMILES: O=c1c2c([nH]c3ccc(Cl)cc13)CCCCC2]

The 2,11-dichloro-7,8,9,10-tetrahydro-6H-cyclohepta[b]quinoline (5.44 g, 20.00 mmol) was dissolved in AcOH (50 mL, 0.4 M) and the reaction mixture was heated in a sealed tube at 200° C. for 24 h. Upon completion, the crude material was poured onto ice and water. The precipitated was filtered, co-evaporated with toluene and the powder was dried in vacuo to afford the desired 2-chloro-5,6,7,8,9,10-hexahydro-11H-cyclohepta[b]quinolin-11-one as orange solid (4.41 g, 17.80 mmol, 89%).

TLC R_{f} (petroleum ether/EtOAc 4:1) 0.07.

IR νₘₐₓ neat (cm⁻¹) 2917, 2848, 1634, 1584, 1550, 1494, 1472, 1455, 1435, 1404, 1362, 1213, 1173.

¹H NMR (400 MHz, DMSO) δ (ppm) 11.57 (br s, 1H, 15), 7.99 (d, J=2.5 Hz, 1H, 2), 7.61 (dd, J=2.5, 8.8 Hz, 1H, 14), 7.52 (d, J=8.9 Hz, 1H, 13), 2.84-2.82 (m, 2H, 10), 2.78-2.75 (m, 2H, 6), 1.82-1.77 (m, 2H, 8), 1.69-1.65 (m, 2H, 7), 1.48-1.43 (m, 2H, 9).

¹³C NMR (126 MHz, DMSO): δ (ppm) 173.61 (4), 153.47 (11), 137.31 (12), 130.97 (14), 127.10 (1), 124.42 (13), 124.23 (2), 121.07 (5), 120.23 (13), 33.56 (10), 31.77 (8), 27.03 (7), 25.64 (9), 23.04 (6).

LRMS (ESI⁺) m/z 248.1 ([M+H]⁺).

HRMS (ESI⁺) m/z calcd for C₁₄H₁₅³⁵ClNO⁺ 248.0847 found 248.0842 Da.

### 2-Chloro-7,8,9,10-tetrahydro-6H-cyclohepta[b]quinolin-1-yl trifluoromethanesulfonate

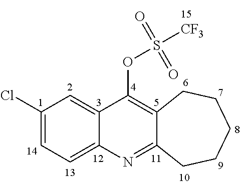 [SMILES: O=S(=O)(Oc1c2c(nc3ccc(Cl)cc13)CCCCC2)C(F)(F)F]

To a suspension of dried triethylamine (2.68 mL, 19.30 mmol, 1.1 equiv) and 2-chloro-5,6,7,8,9,10-hexahydro-11H-cyclohepta[b]quinolin-11-one (4.41 g, 17.80 mmol, 1 equiv) in freshly distilled CH₂Cl₂ (196 mL, 0.1 M), triflic anhydride (3.25 mL, 19.3 mmol, 1.1 equiv) was added dropwise at −78° C. The reaction mixture was stirred 1 h at −78° C. and was then allowed to warm up at room temperature while stirring was continued for 16 h. Upon completion, the solvent was removed under reduced pressure and the remaining dark yellow oil was purified by column chromatography (petroleum ether/EtOAc 9:1) to afford the desired 2-chloro-7,8,9,10-tetrahydro-6H-cyclohepta[b]quinolin-11-yl trifluoromethanesulfonate as orange solid (6.01 g, 16.02 mmol, 90%).

TLC R_{f} (petroleum ether/EtOAc 9:1) 0.28.

MP 88-89° C.

IR νₘₐₓ neat (cm⁻¹) 2929, 2857, 1601, 1479, 1410, 1345, 1323, 1240, 1209, 1134, 1109, 1081.

¹H NMR (400 MHz, CDCl₃) δ (ppm) 7.95 (d, J=9.0 Hz, 1H, 13), 7.90 (d, J=2.2 Hz, 1H, 2), 7.63 (dd, J=2.3, 9.0 Hz, 1H, 14), 3.26-3.23 (m, 2H, 10), 3.04-3.01 (m, 2H, 6), 1.93-1.87 (m, 2H, 9), 1.84-1.76 (m, 4H, 7+8).

¹³C NMR (101 MHz, CDCl₃) δ (ppm) 166.87 (11), 147.48 (4), 146.00 (12), 133.61 (1), 130.92 (14), 130.64 (13), 130.19 (5), 121.75 (3), 120.26 (2), 118.78 (q, J=324.0 Hz, 15), 40.44 (10), 31.83 (8), 27.63 (6), 27.47 (7), 26.71 (9).

¹⁹F-NMR (376 MHz, CDCl₃) δ (ppm) −72.68.

LRMS (ESI⁺) m/z 380.0 ([M+H]⁺).

HRMS (ESI⁺) m/z calcd for C₁₅H₁₄³⁵ClF₃NO₃S⁺ 380.0335 found 380.0332 Da.

### 2-Chloro-N-(prop-2-yn-1-yl)-7,8,9,10-tetrahydro-6H-cyclohepta[b]quinolin-1-amine

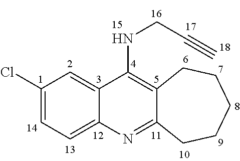 [SMILES: C#CCNc1c2c(nc3ccc(Cl)cc13)CCCCC2]

A microwave tube containing a magnetic stirrer bar was charged with 2-chloro-7,8,9,10-tetrahydro-6H-cyclohepta[b]quinolin-11-yl trifluoromethanesulfonate (100 mg, 0.264 mmol, 1 equiv), Pd₂(dba)₃ (10 mg, 4 mol %), (±)-BINAP (15 mg, 8 mol %) and Cs₂CO₃ (216 mg, 0.660 mmol, 2.5 equiv). The vessel was sealed with a microwave septum and purged with argon. Degassed 1,4-dioxane (3 mL, 9.10⁻² M) and propargylamine (19 μL, 0.343 mmol, 1.3 equiv) were introduced through the septum. The resulting mixture was heated to 100° C. for 1 h using a Biotage® Initiator Microwave Synthesizer Apparatus. After cooling, the reaction mixture was concentrated and purified by column chromatography (Et₂O/hexane 1:1) to afford the desired 2-chloro-N-(prop-2-yn-1-yl)-7,8,9,10-tetrahydro-6H-cyclohepta[b]quinolin-11-amine as yellow wax (63 mg, 0.222 mmol, 84%).

TLC R_{f} (Et₂O/hexane 1:1) 0.27.

IR νₘₐₓ neat (cm⁻¹) 3297, 2922, 2852, 2161, 1587, 1560, 1485, 1440, 1381, 1330, 1160, 1121, 1079.

¹H NMR (400 MHz, CDCl₃) δ (ppm) 7.90 (d, J=2.2 Hz, 1H, 2), 7.88 (d, J=8.9 Hz, 1H, 13), 7.52 (dd, J=8.9, 2.2 Hz, 1H, 14), 4.08-3.92 (m, 3H, 15+16), 3.19-3.16 (m, 2H, 10), 2.98-2.95 (m, 2H, 6), 2.26 (t, J=2.2 Hz, 1H, 18), 1.92-1.86 (m, 2H, 9), 1.82-1.72 (m, 4H, 7+8).

¹³C NMR (101 MHz, CDCl₃) δ (ppm) 165.85 (11), 147.16 (4), 145.48 (12), 131.13 (1), 131.04 (14), 129.28 (13), 127.60 (3), 123.63 (5), 121.67 (2), 80.16 (17), 73.03 (18), 40.34 (16), 39.53 (10), 32.02 (8), 28.52 (6), 27.75 (9), 26.93 (7).

LRMS (ESI⁺) m/z 284.9 ([M+H]⁺).

HRMS (ESI⁺) m/z calcd for C₁₇H₁₈³⁵ClN₂⁺ 285.1153 found 285.1160 Da.

### Methyl 3-(benzyloxy)-6-(3-((2-chloro-7,8,9,10-tetrahydro-6H-cyclohepta[b]quinolin-11-yl)amino)prop-1-yn-1-yl)picolinate

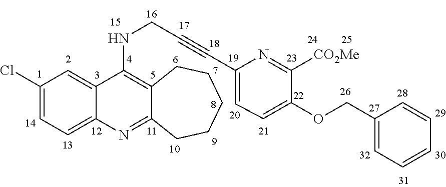 [SMILES: COC(=O)c1nc(C#CCNc2c3c(nc4ccc(Cl)cc24)CCCCC3)ccc1OCc1ccccc1]

To a degassed solution of methyl 3-benzyloxy-6-bromopicolinate (110 mg, 0.341 mmol, 1.1 equiv) in THF/Et₃N (4 mL/3 mL), Pd[PPh₃]₄ (36 mg, 0.031 mmol, 0.1 equiv) and CuI (12 mg, 0.062 mmol, 0.2 equiv) were added. After degassing the reaction mixture 5 min at room temperature, a degassed solution of 2-chloro-N-(prop-2-yn-1-yl)-7,8,9,10-tetrahydro-6H-cyclohepta[h]quinolin-11-amine (118 mg, 0.310 mmol, 1 equiv) in THF (2 mL) was added and the reaction mixture was stirred at the room temperature for 16 h. After completion, the reaction mixture was concentrated under reduced pressure and the residue was purified by column chromatography (petroleum ether/EtOAc 1:1) to afford the desired methyl 3-(benzyloxy)-6-(3-((2-chloro-7,8,9,10-tetrahydro-6H-cyclohepta[b]quinolin-11-yl)amino)prop-1-yn-1-yl)picolinateas yellow wax (145 mg, 0.270 mmol, 87%).

TLC R_{f} (petroleum ether/EtOAc 1:1) 0.21.

IR νₘₐₓ neat (cm⁻¹) 3370, 2922, 2852, 2115, 1730, 1585, 1561, 1485, 1449, 1435, 1380, 1292, 1267, 1209, 1097.

¹H NMR (400 MHz, CDCl₃) δ (ppm) 7.99 (d, J=2.1 Hz, 1H, 2), 7.91 (d, J=8.8 Hz, 1H, 13), 7.53 (dd, J=8.9, 2.0 Hz, 1H, 14), 7.45-7.26 (m, 7H, 20+21+28+29+30+31+32), 5.20 (s, 2H, 26), 4.21 (d, J=6.7, 2H, 16), 4.07 (t, J=6.7 Hz, 1H, 15),3.96 (s, 3H, 25), 3.20-3.17 (m, 2H, 10), 3.02-2.99 (m, 2H, 6), 1.88-1.86 (m, 2H, 9), 1.80-1.74 (m, 4H, 7+8).

¹³C NMR (101 MHz, CDCl₃) δ (ppm) 165.89 (24), 164.71 (11), 153.46 (22), 147.19 (4), 145.41 (12), 140.38 (23), 135.41 (19), 134.13 (27), 131.12 (1), 130.99 (20), 130.13 (14), 129.24 (13), 128.85 (2C, 29+31), 128.38 (30), 128.02 (3), 126.98 (2C, 28+32), 123.87 (5), 121.74 (2), 121.68 (21), 86.21 (18), 83.40 (17), 70.92 (26), 52.83 (25), 40.30 (2C, 10+16), 31.96 (8), 28.34 (6), 27.72 (9), 26.87 (7).

LRMS (ESI⁺) m/z 526.0 ([M+H]⁺).

HRMS (ESI⁺) m/z calcd for C₃₁H₂₉³⁵ClN₃O₃⁺ 526.1892 found 526.1893 Da.

### Methyl 3-hydroxy-6-(3-((7,8,9,10-tetrahydro-6H-cyclohepta[b]quinolin-11-yl)amino)propyl)picolinate

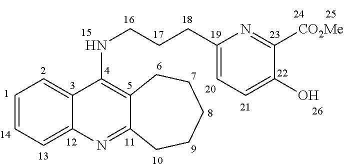 [SMILES: COC(=O)c1nc(CCCNc2c3c(nc4ccccc24)CCCCC3)ccc1O]

To a degassed solution of methyl 3-(benzyloxy)-6-(3-((2-chloro-7,8,9,10-tetrahydro-6H-cyclohepta[b]quinolin-11-yl)amino)prop-1-yn-1-yl)picolinate (100 mg, 0.190 mmol, 1 equiv) in dry EtOH (10 mL, 0.02 M), Pearlman's catalyst Pd(OH)₂/C (20% with 50% moisture, 80 mg, 0.057 mmol, 0.3 equiv) was added. After evaporating and flushing with H₂ five times, the reaction mixture was stirred 1 h at room temperature under H₂ (1 atm.). Upon completion, the catalyst was removed by filtration through a short column of celite, the solvent was evaporated, and the residue was purified by column chromatography (CH₂Cl₂/MeOH 9:1) to afford methyl 3-hydroxy-6-(3-((7,8,9,10-tetrahydro-6H-cyclohepta[b]quinolin-11-yl)amino)propyl)picolinate as white wax (69 mg, 0.171 mmol, 90%).

TLC R_{f} (CH₂Cl₂/MeOH 9:1) 0.32.

IR νₘₐₓ neat (cm⁻¹) 3261, 2942, 2854, 1674, 1634, 1584, 1566, 1467, 1445, 1362, 1302, 1212, 1101.

¹H NMR (400 MHz, CDCl₃) δ (ppm) 10.46 (br s, 1H, 26), 8.32 (d, J=8.3 Hz, 1H, 2), 8.22 (d, J=8.6 Hz, 1H, 13), 7.58 (t, J=7.7 Hz, 1H, 14), 7.38 (t, J 7.7 Hz, 1H, 1), 7.32-7.22 (m, 2H, 20+21), 6.46 (br s, 1H, 15), 3.95 (s, 3H, 25), 3.62 (t, J=6.7 Hz, 2H, 16), 3.38-3.25 (m, 2H, 10), 2.94-2.80 (m, 4H, 6+18), 2.17 (quin, J=7.0 Hz, 2H, 17), 1.90-1.74 (m, 4H, 7+9), 1.70-1.57 (m, 2H, 8).

¹³C NMR (101 MHz, CDCl₃): δ (ppm) 169.87 (24), 161.26 (11), 157.44 (19), 153.82 (4), 152.35 (22), 140.48 (12), 130.76 (23), 129.60 (14), 128.88 (20), 127.19 (13), 125.69 (1), 123.66 (21), 122.91 (2), 119.33 (3), 118.92 (5), 53.22 (25), 48.12 (16), 35.71 (18), 34.17 (10), 31.53 (8), 30.48 (6), 27.61 (9), 27.42 (17), 26.24 (7).

LRMS (ESI⁺) m/z 406.2 ([M+H]⁺).

HRMS (ESI⁺) m/z calcd for C₂₄H₂₈N₂O₃⁺ 3406.2125 found 406.2127 Da.

### 3-Hydroxy-6-(3-((7,8,9,10-tetrahydro-6H-cyclohepta[b]quinolin-11-yl)amino)propyl)picolinaldehyde

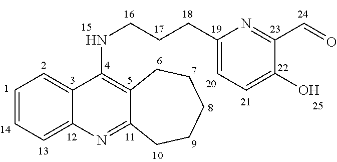 [SMILES: O=Cc1nc(CCCNc2c3c(nc4ccccc24)CCCCC3)ccc1O]

A solution of methyl 3-hydroxy-6-(3-((7,8,9,10-tetrahydro-6H-cyclohepta[b]quinolin-11-yl)amino)propyl)picolinate (15 mg, 0.037 mmol, 1 equiv), imidazole (10 mg, 0.148 mmol, 4 equiv), and tert-butyldimethylsilyl chloride (12 mg, 0.081 mmol, 2.2 equiv) in dry DMF (1.1 mL, 0.03 M) was stirred at the room temperature under argon atmosphere during 2 h. After completion, ethyl acetate was added, and the organic phase was washed with water thrice, dried over Na₂SO₄ and concentrated under reduced pressure.

LRMS (ESI⁺) m/z 520.1 [M+H]⁺.

After drying in vacuo, the residue was subjected to the following step. To the solution of methyl 3-((tert-butyldimethylsilyl)oxy)-6-(3-((7,8,9,10-tetrahydro-6H-cyclohepta[b]quinolin-11-yl)amino)propyl)picolinate (18 mg, 0.034 mmol, 1 equiv) in dry CH₂Cl₂ (1 mL, 0.03 M) at −78° C., DIBAL-H (1M solution in CH₂Cl₂, 62 μL, 0.062 mmol, 2 equiv) was added dropwise. The reaction mixture was stirred at −78° C. for 12 min, then the reaction was quenched with MeOH (2 mL), and the cooling bath was removed. When the mixture was warmed to room temperature the organic phase was washed with aqueous solution of NaOH (1M), dried over Na₂SO₄ and concentrated under reduced pressure. LRMS (ESI⁺) m/z 490.1 [M+H]⁺.

After drying in vacuo, the residue was subjected to the following step. To the solution of 3-((tert-butyldimethylsilyl)oxy)-6-(3-((7,8,9,10-tetrahydro-6H-cyclohepta[b]quinolin-11-yl)amino)propyl)picolinaldehyde (13 mg, 0.027 mmol, 1 equiv) in dry THF (1 mL, 0.03 M) at 0° C., TBAF (1M solution in THF, 30 μL, 0.030 mmol, 1.1 equiv) was added, and the reaction mixture was stirred at 0° C. for 1 h. After completion, the solvent was removed under reduced pressure and the crude material was purified by column chromatography (CH₂C₂/MeOH 95:5 to 9:1) to afford the desired 3-hydroxy-6-(3-((7,8,9,10-tetrahydro-6H-cyclohepta[b]quinolin-11-yl)amino)propyl)picolinaldehyde as bright yellow wax (10 mg, 0.027 mmol, 71% over 3 steps).

TLC R_{f} (CH₂Cl₂/MeOH 9/1) 0.3.

IR νₘₐₓ neat (cm⁻¹) 3279, 2922, 2855, 1660, 1585, 1566, 1497, 1464, 1427, 1348, 1287, 1154.

¹H NMR (400 MHz, CDCl₃) δ (ppm) 10.01 (s, 1H, 24), 8.16 (d, J=8.2 Hz, 1H, 13), 7.98 (d, J=8.4 Hz, 1H, 2), 7.60 (t, J=7.6 Hz, 1H, 14), 7.42 (d, J=7.8 Hz, 1H, 1), 7.29 (m, 2H, 20+21), 3.51 (t, J=6.7 Hz, 2H, 16), 3.33-3.19 (m, 2H, 10), 2.97-2.83 (m, 4H, 6+18), 2.17 (quin, J=7.1 Hz, 2H, 17), 1.91-1.75 (m, 4H, 7+9), 1.72-1.62 (m, 2H, 8). ¹³C NMR (101 MHz, CDCl₃) δ (ppm) 198.49 (24), 157.32 (19), 153.48 (22), 135.86 (14), 130.06 (20), 126.93 (21), 125.45 (1), 122.28 (2+13), 49.19 (16), 34.31 (18), 31.83 (10), 30.70 (8), 29.84 (6), 27.90 (9), 27.69 (17), 26.61 (7). Short run was made due to the stability of aldehyde, thus some quaternary carbons were not visible (3, 4, 5, 11, 23 and 12)

LRMS (ESI⁺) m/z 375.9 ([M+H]⁺).

HRMS (ESI⁺) m/z calcd for C₂₃H₂₆N₃O₂ 376.2021 found 376.2020 Da.

### 3-Hydroxy-6-(3-((7,8,9,10-tetrahydro-6H-cyclohepta[b]quinolin-11-yl)amino)propyl)picolinaldehyde oxime

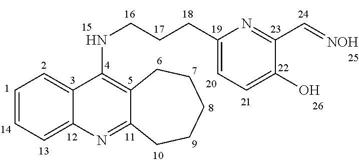

A solution of 3-hydroxy-6-(3-((7,8,9,10-tetrahydro-6H-cyclohepta[b]quinolin-11-yl)amino)propyl)picolinaldehyde (10 mg, 0.027 mmol, 1 equiv), hydroxylamine hydrochloride (3 mg, 0.041 mmol, 1.5 equiv), and CH₃CO₂Na (3.4 mg, 0.041 mmol, 1.5 equiv) in dry ethanol (1 mL, 0.03 M) was stirred at 40° C. during 16 h. After concentration under reduced pressure, the crude product was purified by the column chromatography (CH₂Cl₂/MeOH 9:1) to afford the desired 3-hydroxy-6-(3-((7,8,9,10-tetrahydro-6H-cyclohepta[b]quinolin-11-yl)amino)propyl)picolinaldehyde oxime as bright yellow wax (9 mg, 0.023 mmol, 87%).

TLC R_{f} (CH₂Cl₂/MeOH 9:1) 0.26.

IR νₘₐₓ neat (cm⁻¹) 3262, 2924, 2854, 1635, 1585, 1522, 1464, 1427, 1271, 1167, 1021.

¹H NMR (400 MHz, CD₃OD) δ (ppm) 8.23 (d, J=8.6 Hz, 1H, 13), 8.05 (s, 1H, 24), 7.84-7.74 (m, 2H, 2+14), 7.56 (m, 1H, 1), 7.18 (d, J=8.4 Hz, 1H, 21), 7.06 (d, J=8.4 Hz, 1H, 20), 3.76 (t, J=6.7 Hz, 2H, 16), 3.21-3.05 (m 2H, 10), 2.96-2.86 (m, 2H, 6), 2.82 (t, J=7.0 Hz, 2H, 18), 2.26-2.15 (m, 2H, 17), 1.98-1.87 (m, 3H, 15+7), 1.83-1.81 (m, 2H, 7), 1.69-1.57 (m, 2H, 8).

¹³C NMR (101 MHz, CD₃OD) δ (ppm) 160.60 (11), 157.47 (19), 153.96 (22), 153.20 (4), 152.86 (24), 139.60 (12), 136.62 (23), 133.46 (14), 127.21 (13), 126.09 (1), 125.56 (20), 125.12 (21), 121.45 (2), 119.40 (3), 118.54 (5), 35.85 (16), 34.89 (10), 32.41 (8), 31.34 (18), 31.05 (17), 28.20 (6), 28.06 (9), 27.10 (7).

LRMS (ESI⁺) m/z 391.2 ([M+H]⁺).

HRMS (ESI⁺) m/z calcd for C₂₃H₂₇N₄O₂⁺391.2129 found 391.2136 Da.

### 1.13—Synthesis of Compound 18

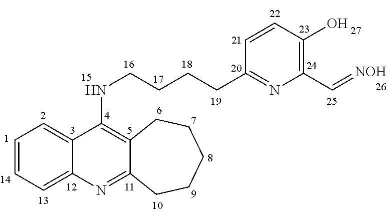

### N-(But-3-yn-1-yl)-2-chloro-7,8,9,10-tetrahydro-6H-cyclohepta[b]quinolin-11-amine

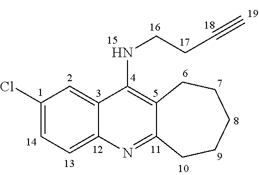 [SMILES: C#CCCNc1c2c(nc3ccc(Cl)cc13)CCCCC2]

A microwave tube containing a magnetic stirrer bar was charged with 2-chloro-7,8,9,10-tetrahydro-6H-cyclohepta[b]quinolin-1 l-yl trifluoromethanesulfonate (100 mg, 0.264 mmol, 1 equiv), Pd₂(dba)₃ (10 mg, 4 mol %), (±)-BINAP (14 mg, 8 mol %) and Cs₂CO₃ (216 mg, 0.660 mmol, 2.5 equiv). The vessel was sealed with a microwave septum and purged with argon. Degassed 1,4-dioxane (3 mL, 9.10⁻² M) and 1-amino-3-butyne (28 μL, 0.343 mmol, 1.3 equiv) were introduced through the septum. The resulting mixture was heated to 100° C. for 1 h using a Biotage® Initiator Microwave Synthesizer Apparatus. After cooling, the reaction mixture was concentrated and purified by column chromatography (Et₂O/hexane 1:1) to afford the desired N-(but-3-yn-1-yl)-2-chloro-7,8,9,10-tetrahydro-6H-cyclohepta[b]quinolin-11-amine as yellow solid (73 mg, 0.244 mmol, 93%).

TLC R_{f} (Et₂O/hexane 1:1) 0.26.

MP97-98° C.

IR νₘₐₓ neat (cm⁻¹) 3299, 2921, 2851, 2118, 1584, 1560, 1486, 1452, 1440, 1425, 1384, 1346, 1199, 1150, 1121, 1082.

¹H NMR (400 MHz, CDCl₃) δ (ppm) 7.99 (d, J=2.2 Hz, 1H, 2), 7.87 (d, J=9.1 Hz, 1H, 13), 7.51 (dd, J=2.2, 9.1 Hz, 1H, 14), 4.14 (br s, 1H, 15), 3.40 (q, J=6.1 Hz, 2H, 16), 3.24-3.09 (m, 2H, 10), 3.05-2.87 (m, 2H, 6), 2.45 (dt, J=2.7, 6.1 Hz, 2H, 17), 2.18 (t, J=2.6 Hz, 1H, 19), 1.94-1.83 (m, 2H, 9), 1.83-1.65 (m, 4H, 7+8).

¹³C NMR (101 MHz, CDCl₃) δ (ppm) 165.82 (11), 147.87 (4), 145.46 (12), 130.96 (14), 130.79 (1), 129.12 (13), 126.67 (3), 123.62 (5), 121.67 (2), 81.86 (18), 71.09 (19), 48.67 (16), 40.27 (10), 32.03 (8), 28.37 (6), 27.63 (9), 26.92 (7), 20.68 (17).

LRMS (ESI⁺) m/z 299.1 ([M+H]⁺).

HRMS (ESI⁺) m/z calcd for C₁₈H₂₀³⁵ClN₂⁺ 299.1310 found 299.1307 Da.

### Methyl 3-(benzyloxy)-6-(4-((2-chloro-7,8,9,10-tetrahydro-6H-cyclohepta[b]quinolin-1 l-yl)amino)but-1-yn-1-yl)picolinate

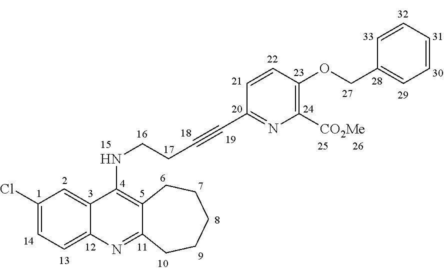 [SMILES: COC(=O)c1nc(C#CCCNc2c3c(nc4ccc(Cl)cc24)CCCCC3)ccc1OCc1ccccc1]

To a degassed solution of methyl 3-benzyloxy-6-bromopicolinate (320 mg, 0.994 mmol, 1.1 equiv) in THF/Et₃N (8 mL/5 mL), Pd[PPh₃]₄ (104 mg, 0.090 mmol, 0.1 equiv) and CuI (34 mg, 0.181 mmol, 0.2 equiv) were added. After degassing the reaction mixture 5 min at room temperature, a degassed solution of N-(but-3-yn-1-yl)-2-chloro-7,8,9,10-tetrahydro-6H-cyclohepta[b]quinolin-11-amine (270 mg, 0.904 mmol, 1 equiv) in THF (2 mL) was added and the reaction mixture was stirred at the room temperature for 16 h. After completion, the reaction mixture was concentrated under reduced pressure and the residue was purified by column chromatography (EtOAc/hexane 7:3) to afford the desired methyl 3-(benzyloxy)-6-(4-((2-chloro-7,8,9,10-tetrahydro-6H-cyclohepta[b]quinolin-11-yl)amino)but-1-yn-1-yl)picolinate as yellow wax (464 mg, 0.859 mmol, 95%).

TLC R_{f} (EtOAc/hexane 7:3) 0.33.

IR νₘₐₓ neat (cm⁻¹) 3376, 3062, 2922, 2851, 2229, 1733, 1584, 1561, 1487, 1450, 1435, 1383, 1291, 1270, 1209, 1150, 1098.

¹H NMR (400 MHz, CDCl₃) δ (ppm) 7.98 (d, J=2.0 Hz, 1H, 2), 7.86 (d, J=8.8 Hz, 1H, 13), 7.49 (dd, J=2.1, 8.9 Hz, 1H, 14), 7.46-7.27 (m, 7H, 21+22+29+30+31+32+33), 5.21 (s, 2H, 27), 4.18 (br s, 1H, 15), 3.96 (s, 3H, 26), 3.56-3.42 (m, 2H, 16), 3.24-3.06 (m, 2H, 10), 3.03-2.90 (m, 2H, 6), 2.66 (t, J=6.1 Hz, 2H, 17), 1.92-1.82 (m, 2H, 9), 1.82-1.66 (m, 4H, 7+8).

¹³C NMR (101 MHz, CDCl₃) δ (ppm) 165.89 (25), 164.86 (11), 153.30 (23), 147.89 (4), 145.39 (12), 140.46 (24), 135.52 (20), 134.73 (28), 130.94 (21), 130.78 (1), 130.14 (14), 129.12 (13), 128.85 (30+32), 128.37 (31), 127.01 (29+33), 126.53 (3), 123.57 (5), 121.82 (2), 121.55 (22), 87.09 (18), 81.75 (19), 70.96 (27), 52.80 (26), 48.50 (16), 40.28 (10), 32.02 (8), 28.30 (6), 27.78 (9), 26.89 (7), 21.67 (17).

LRMS (ESI⁺) m/z 540.1 ([M+H]⁺).

HRMS (ESI⁺) m/z calcd for C₃₂H₃₁³⁵ClN₃O₃⁺ 540.2048 found 540.2049 Da.

### Methyl 3-hydroxy-6-(4-((7,8,9,10-tetrahydro-6H-cyclohepta[b]quinolin-11-yl) amino)butyl)picolinate

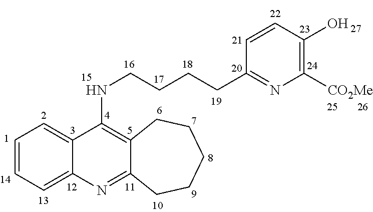 [SMILES: COC(=O)c1nc(CCCCNc2c3c(nc4ccccc24)CCCCC3)ccc1O]

To a degassed solution of methyl 3-(benzyloxy)-6-(4-((2-chloro-7,8,9,10-tetrahydro-6H-cyclohepta[b]quinolin-11-yl)amino)but-1-yn-1-yl) picolinate (60 mg, 0.111 mmol) in EtOH (10 mL, 0.01 M), Pearlman's catalyst Pd(OH)₂/C (20% with 50% moisture, 47 mg, 0.033 mmol, 0.3 equiv) was added. After evaporating and flushing with H₂ five times, the reaction mixture was stirred 1 h at room temperature under H₂ (1 atm.). Upon completion, the catalyst was removed by filtration through a short column of celite, the solvent was evaporated and the residue was purified by column chromatography (CH₂Cl₂/MeOH 9:1) to afford the desired methyl 3-hydroxy-6-(4-((7,8,9,10-tetrahydro-6H-cyclohepta[b]quinolin-11-yl)amino)butyl)picolinate as white wax (42 mg, 0.100 mmol, 90%).

TLC R_{f} (CH₂Cl₂/MeOH 9:1) 0.26.

IR νₘₐₓ neat (cm⁻¹) 3336, 2921, 2852, 1728, 1673, 1585, 1563, 1498, 1466, 1444, 1369, 1298, 1205, 1100.

¹H NMR (500 MHz, CDCl₃) δ (ppm) 10.60 (br s, 1H, 27), 8.02 (d, J=7.6 Hz, 1H, 2), 7.89 (d, J=8.3 Hz, 1H, 13), 7.58 (dt, J=1.1, 9.0 Hz, 1H, 14), 7.40 (ddd, J=1.2, 7.0, 8.4 Hz, 1H, 1), 7.34-7.19 (m, 2H, 21+22), 4.01 (s, 3H, 26), 3.34 (t, J=6.8 Hz, 2H, 16), 3.27-3.13 (m, 2H, 10), 2.96-2.86 (m, 2H, 6), 2.82 (t, J=7.6 Hz, 2H, 19), 2.04-1.59 (m, 10H, 7+8+9+17+18).

¹³C NMR (126 MHz, CDCl₃) δ (ppm) 170.15 (25), 164.84 (11), 157.36 (20), 153.38 (23), 150.31 (4), 145.92 (12), 129.25 (14), 128.99 (21), 128.69 (22), 128.45 (24), 126.84 (13), 124.95 (1), 123.49 (3), 122.07 (2), 121.79 (5), 53.23 (26), 50.40 (16), 39.52 (19), 37.18 (10), 32.01 (8), 31.01 (6), 28.27 (9), 27.72 (17), 27.32 (7), 26.87 (18).

LRMS (ESI⁺) m/z 420.1 ([M+H]⁺).

HRMS (ESI⁺) m/z calcd for C₂₅H₃₀N₃O₃⁺ 420.2282 found 420.2282 Da.

### 3-Hydroxy-6-(4-((7,8,9,10-tetrahydro-6H-cyclohepta[b]quinolin-11-yl)amino)butyl)picolinaldehyde

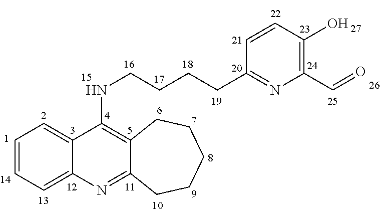 [SMILES: O=Cc1nc(CCCCNc2c3c(nc4ccccc24)CCCCC3)ccc1O]

To a solution of methyl 3-hydroxy-6-(4-((7,8,9,10-tetrahydro-6H-cyclohepta[b]quinolin-11-yl)amino)butyl)picolinate (60 mg, 0.143 mmol, 1 equiv) in dry CH₂Cl₂ (1.5 mL, 0.1 M) were successively added dry 2,6-lutidine (50 μL, 0.429 mmol, 3 equiv) and trimethylsilyl trifluoromethanesulfonate (99 μL, 0.429 mmol, 3 equiv) at room temperature. The reaction mixture was stirred at room temperature for 4 h under argon atmosphere. After completion, the mixture was washed with saturated solution of CuSO₄, brine, dried over Na₂SO₄, filtered and concentrated under reduced pressure. LRMS (ESI⁺) m/z 534.2 [M+H]⁺.

After drying in vacuo, the residue was subjected to the following step. To the solution of methyl 3-((tert-butyldimethylsilyl)oxy)-6-(4-((7,8,9,10-tetrahydro-6H-cyclohepta[b]quinolin-11-yl)amino)butyl)picolinate (76 mg, 0.143 mmol, 1 equiv) in dry CH₂Cl₂ (1.5 mL, 0.1 M) at −78° C., DIBAL-H (1M solution in CH₂Cl₂, 429 μL, 0.429 mmol, 3 equiv) was added dropwise. The reaction mixture was stirred at −78° C. for 12 min, then the reaction was quenched with MeOH (500 μL), and the cooling bath was removed. When the mixture was warmed to room temperature the organic phase was washed with aqueous solution of NaOH (1M), dried over Na₂SO₄ and concentrated under reduced pressure. LRMS (ESI⁺) m/z 504.3 [M+H]⁺.

After drying in vacuo, the residue was subjected to the following step. To the solution of 3-((tert-butyldimethylsilyl)oxy)-6-(4-((7,8,9,10-tetrahydro-6H-cyclohepta[b]quinolin-11-yl)amino)butyl)picolinaldehyde (72 mg, 0.143 mmol, 1 equiv) in dry THF (1.5 mL, 0.1 M) at 0° C., TBAF (1M solution in THF, 157 μL, 0.157 mmol, 1.1 equiv) was added, and the reaction mixture was stirred at 0° C. for 2 h. After completion, the solvent was removed under reduced pressure and the crude material was purified by column chromatography (CH₂Cl₂/MeOH 95:5 to 9:1) to afford the desired 3-hydroxy-6-(4-((7,8,9,10-tetrahydro-6H-cyclohepta[b]quinolin-11-yl)amino)butyl)picolinaldehyde as bright yellow wax (25 mg, 0.064 mmol, 45% over 3 steps).

TLC R_{f} (CH₂Cl₂/MeOH 9/1) 0.27.

IR νₘₐₓ neat (cm⁻¹) 3313, 2921, 2852, 1661, 1585, 1567, 1464, 1353, 1285, 1260, 1212, 1153, 1090, 1019.

¹H NMR (400 MHz, CDCl₃) δ (ppm) 9.99 (s, 1H, 25), 8.13 (d, J=8.3 Hz, 1H, 2), 8.05-7.90 (m, 1H, 3), 7.58 (t, J=7.0 Hz, 1H, 14), 7.41 (t, J=7.7 Hz, 1H, 1), 7.33-7.19 (m, 2H, 21+22), 3.49 (t, J=6.5 Hz, 2H, 16), 3.33-3.14 (m, 2H, 10), 2.92-2.85 (m, 2H, 6), 2.81 (t, J=7.1 Hz, 2H, 19), 1.90-1.53 (m, 10H, 7+8+9+17+18).

¹³C NMR (101 MHz, CDCl₃) δ (ppm) 198.68 (25), 163.09 (11), 157.19 (20), 154.21 (23), 151.88 (4), 143.34 (12), 135.88 (24), 129.89 (21), 129.77 (14), 126.65 (22), 125.37 (13), 122.47 (1), 122.42 (2), 121.40 (3), 120.55 (5), 49.91 (16), 36.84 (19), 31.81 (10), 30.93 (8), 29.81 (6), 27.91 (9), 27.62 (17), 26.88 (18), 26.59 (7).

LRMS (ESI⁺) m/z 390.2 ([M+H]⁺).

HRMS (ESI⁺) m/z calcd for C₂₄H₂₈N₃O₂⁺ 390.2176 found 390.2182 Da.

### 3-Hydroxy-6-(4-((7,8,9,10-tetrahydro-6H-cyclohepta[b]quinolin-11-yl)amino)butyl)picolinaldehyde oxime

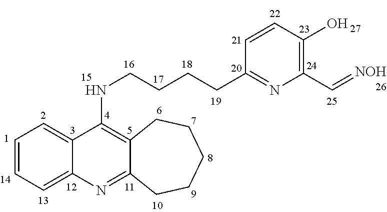

A solution of 3-hydroxy-6-(4-((7,8,9,10-tetrahydro-6H-cyclohepta[b]quinolin-11-yl)amino)butyl)picolinaldehyde (20 mg, 0.051 mmol, 1 equiv), hydroxylamine hydrochloride (5 mg, 0.077 mmol, 1.5 equiv), and anhydrous CH₃CO₂Na (6 mg, 0.077 mmol, 1.5 equiv) in dry ethanol (1 mL, 0.05 M) was stirred at reflux during 15 h. After concentration under reduced pressure, the crude material was purified by the column chromatography (CH₂Cl₂/MeOH 9:1) to afford 3-hydroxy-6-(4-((7,8,9,10-tetrahydro-6H-cyclohepta[b]quinolin-11-yl)amino)butyl)picolinaldehyde oxime as yellow wax (15 mg, 0.037 mmol, 73%).

TLC R^{f} (CH₂Cl₂/MeOH 9:1) 0.19.

IR νₘₐₓ neat (cm⁻¹) 3269, 2923, 2854, 1634, 1584, 1463, 1427, 1354, 1270, 1198, 1165, 1119, 1022, 965.

¹H NMR (500 MHz, CDCl₃) δ (ppm) 8.25 (d, J=8.6 Hz, 1H, 2), 8.13 (s, 1H, 25), 7.82-7.76 (m, 2H, 13+14), 7.61-7.51 (m, 1H, 1), 7.18 (d, J=8.4 Hz, 1H, 22), 7.06 (d, J=8.4 Hz, 1H, 21), 3.73 (t, J=6.5 Hz, 2H, 16), 3.18-3.10 (m, 2H, 10), 2.98-2.88 (m, 2H, 6), 2.72 (t, J=6.7 Hz, 2H, 19), 1.99-1.88 (m, 3H, 17+18a), 1.85-1.66 (m, 7H, 7+8+9+18b).

¹³C NMR (126 MHz, CDCl₃) δ (ppm) 160.51 (11), 156.95 (20), 154.10 (23), 153.72 (4), 152.75 (25), 139.92 (12), 136.29 (24), 133.17 (14), 126.98 (22), 125.89 (13), 125.36 (21), 125.19 (1), 121.66 (2), 119.38 (3), 119.00 (5), 49.85 (16), 37.12 (19), 35.82 (10), 32.30 (8), 30.92 (6), 28.05 (17), 27.86 (9), 27.62 (7), 27.00 (18).

LRMS (ESI⁺) m/z 405.1 ([M+H]⁺).

### Example 2

### Docking and Molecular Dynamics Simulations

### Materials and Methods

### Flexible Docking Conditions:

Dockings have been performed using autodock vina (Ref http://onlinelibrary.wiley.com/doi/10.1002/jcc.21334/abstract) and by preparing the system in PyMOL (Schrödinger) using the plug-in developed by Daniel Seeliger (http://wwwuser.gwdg.de/˜dseelig/adplugin.html). VX-hAChE was constructed from the apo form (pdb code 4EY4) by homology to the mAChE-VX structure (pdb code 2Y2U), keeping in the active site all the usually conserved water molecules. Residues in the gorge (Tyr72, Asp74, Trp86, Tyr124, Ser125, Trp286, Tyr337. Phe338, Tyr341) have been chosen as flexible, along with the ethyl group of VX. A docking box of 60×60×60 Angstroms was chosen, centered at the bottom of the gorge between Tyr124 and Trp86. Ligands were built and optimized from SMILEs string using Phenix elbow (Ref http://scripts.iucr.org/cgi-bin/paper?dz5186). The default parameter set of Autodock vina was used to generate 9 docking poses per molecule.

### MD Simulations

Molecular dynamics simulations were carried out using GROMACS 4.5.6 [Hess, B.; Kutzner, C.; van der Spoel, D.; Lindahl, E. J. Chem. Theory Comput. 2008, 4, 435-447.] and the Amber99sb force field. [Hornak, V.; Abel, R.; Okur, A.; Strockbine, B.; Roitberg, A.; Simmerling, C. Proteins 2006, 65, 712-725]. The topological description of each KM molecule was built using acpype and the general amber force field [Wang, J.; Wolf, R. M.; Caldwell, J. W.; Kollman, P. A.; Case, D. A. J. Comput. Chem. 2004, 25, 1157-1174.] The hAChE-VX complex in the conformation obtained from flexible dockings together with crystal water molecules and the different KM molecules, was immersed in a periodic water box of cubic shape with a minimal distance of 10 Å to any edge and periodic boundary conditions. The box was solvated using the TIP3P solvation model and chloride and sodium counter ions were added to neutralize the simulation system. After energy minimization using a 500-step steepest decent method, the system was subjected to equilibration at 1 bar for 50 ps under the conditions of position restraints for heavy atoms. The solvent, the counter ions, and the protein were coupled separately to a temperature bath at 300 K. The Lennard-Jones interactions were cut off at 1.4 nm. The long-range electrostatic interactions were handled using particle-mesh Ewald method for determining long-range electrostatics (9 Å cutoff). Temperature was set to 300 K and was kept constant using a Berendsen thermostat [Berendsen, H. J. C.; Postma, J. P. M.; van Gunsteren, W. F.; DiNola, A.; Haak, J. R. J. Chem. Phys. 1984, 81, 3684](with a coupling time constant of 0.1 ps). Pressure with a reference value of 1 bar was controlled by a Berendsen barostat (with a coupling time constant of 1 ps and a compressibility of 4.5 10⁵ bar). Full MD simulation was performed for 5 ns at 300 K, using 2 femtosecond timesteps. All bond lengths were constrained using the LINCS algorithm allowing an integration step of 2 fs [Hess, B.; Bekker, H.; Berendsen, H. J. C.; Fraaije, J. G. E. M. J. Comput. Chem. 1997, 18, 1463]. Coordinates were saved every 250 steps (i.e. every 0.5 ps).

### Results

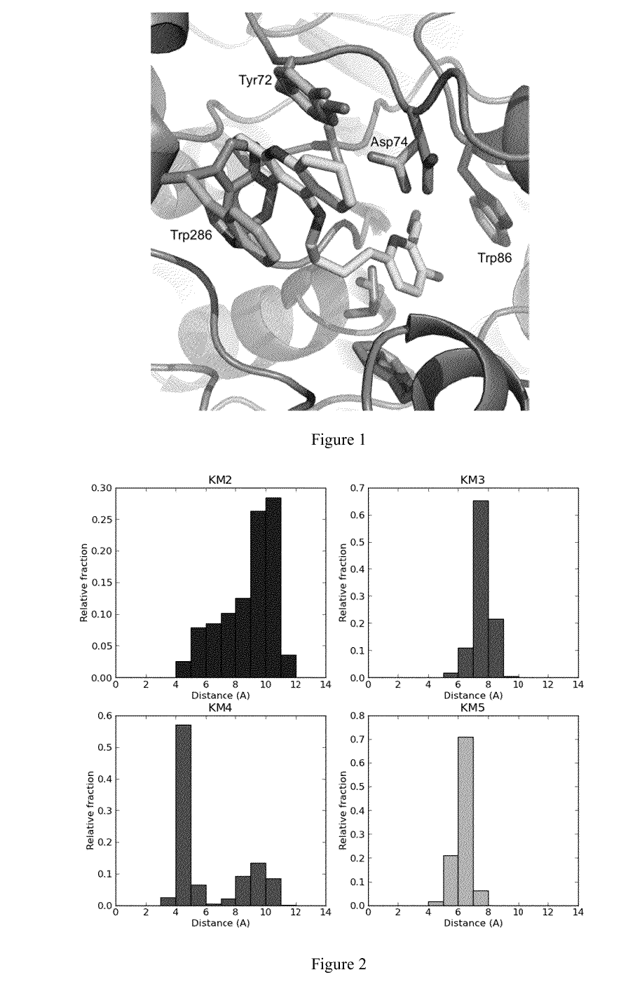

Ternary complexes between VX-inhibited human acetylcholine esterase (hAChE) and 3-hydroxypyridine aldoxime 11 (5 carbons linker) and 12 (4 carbons linker) and derivatives with a shorter linker (3 and 2 carbons) have been studied by molecular docking and molecular dynamics simulations. Flexible docking experiments have been performed by allowing side-chain rotation of some key residues in the gorge starting from their native conformation. Each of the four docking results (FIG. 1) indicated a conformational change of the peripheral-site Trp286 similar to the one seen in a bis-tacrine/TcAChE complex (PDB code 2CEK). The tacrine moiety is found to form a π-π stacking sandwich in between Tyr72 and Trp286. The scoring function yields similar binding energies for the 4 molecules (about −9 kcal/mol), suggesting that the linker is sufficiently long to prevent unsuited accomodation of the molecule in the gorge.

The conformation resulting from the four dockings have then been equilibrated for 5 ns at a temperature of 300 K in molecular dynamics (MD) simulations. The distance between the aldoxime oxygen and the VX-AChE phosphorus atom was monitored along the simulation trajectory and the distribution of distances was calculated (FIG. 2). A distance shorter than 4 Å means that the two atoms are sufficiently close to enhance the rate of formation of a covalent bond, i.e. that reactivation may occur at a fast rate.

Molecule 12 brings the aldoxime oxygen both closer to the phosphorus atom and more frequently than the other three molecules, indicating that a 4-carbon linker is optimum for reactivation.

### Example 3

### Reactivation of hAChE Inhibited by OPNAs

### Materials and Methods

IC₅₀ measurements. Recombinant hAChE was produced and purified as previously described [Carletti et al 2008 J Am Chem Soc 130(47):16011-20). Oximes were dissolved in MeOH to make 5- or 10-mM stock solution and further diluted in phosphate buffer (sodium phosphate 0.1 M, pH 7.4). Recombinant hAChE activity was measured spectrophotometrically (absorbance at 412 nm) in the presence of various concentrations of oximes in 1 mL Ellman's buffer (sodium phosphate 0.1 M, pH 7.4, 0.1% BSA, 0.5 mM DTNB, 25° C.). Measurements were performed at least in duplicate for each concentration tested. The concentration of oxime producing 50% of enzyme inhibition was determined by non-linear fitting using ProFit (Quantumsoft) using the standard IC₅₀ equation: % Activity=100*IC₅₀/(IC₅₀+[Ox]).

Inhibition of hAChE by OPNAs.

Recombinant hAChE was produced and purified as previously described (see reference: http://www.ncbi.nlm.nih.gov/pubmed/18975951) VX and tabun were from DGA maîtrise NRBC (Vert le Petit, France). Paraoxon-ethyl was purchased from Sigma-Aldrich. HI-6 was from Pharmacie Centrale des Armées (Orléans, France). All other chemicals including paraoxon were from Sigma. Stock solution of VX and tabun were 5 mM in isopropanol. The inhibition of 120 μM hAChE was carried out with a 5-fold excess of OPNAs and was performed in tris buffer (20 mM, pH 7.4, 0.1% BSA) at 25° C. After a 20-minute incubation, inhibited hAChE was desalted on PD-10 column (GE Healthcare).

Reactivation of hAChE Inhibited by OPNAs.

OPNA-inhibited hAChE was incubated at 37° C. with at least 4 or 5 concentrations of oxime in phosphate buffer (0.1 M, pH 7.4, 0.1% BSA). The final concentration of MeOH in the incubation mix was below 2% and had no influence on the enzyme stability. At time intervals ranging from 1 to 10 minutes depending on the reactivation rate, 10-μL aliquots of each solutions containing the different concentrations of oxime were transferred to cuvettes containing 1 mM acetylthiocholine in 1 mL Ellman's buffer (phosphate 0.1 M, pH 7.4, 0.1% BSA, 0.5 mM DTNB, 25° C.) for measurement of hAChE activity.

The enzyme activity in the control remained constant during the experiment. The percentage of reactivated enzyme (% E_{react}) was calculated as the ratio of the recovered enzyme activity and activity in the control. The apparent reactivation rate k_{obs} for each oxime concentration, the dissociation constant K_{D} of inhibited enzyme-oxime complex (E-POx) and the maximal reactivation rate constant kᵣ, were calculated by non-linear fit with ProFit (Quantumsoft) using the standard oxime concentration-dependent reactivation equation derived from the following scheme:

$$E \text{-} P + Ox {⇄}^{{K}_{D}} E \text{-} POx {\to }^{{k}_{r}} E + Pox$$

$$\% {E}_{react} = 100 \cdot ( 1 - {e}^{{k}_{obs} \cdot t} ) and {k}_{obs} = \frac{{k}_{r} [ Ox ]}{{K}_{D} + [ Ox ]}$$

### Results

The ability of amidoximes 6, 8 and 9 and aldoximes 11, 12 and 15 to reactivate in vitro VX, tabun and/or ethyl paraoxon-hAChE was studied by spectrophotometry using Ellman's reagent and compared to known reactivators such as 2-PAM, obidoxime, HLö-7, trimedoxime (TMB4) and HI-6.

The results are provided in tables 1 and 2 below.

TABLE 1
In vitro Reactivation of VX-hAChE

| | kᵣ | K_{D} | kᵣ₂ |
|---|---|---|---|
| Reactivator | (min⁻¹) | (μM) | (mM⁻¹min⁻¹)^{b} |
| 2-PAM | 0.06 ± 0.01 | 215 ± 75 | 0.28 |
| Obidoxime | 0.60 ± 0.05 | 54 ± 12 | 11 |
| HLö-7 | 0.49^{a} | 7.8^{a} | 63^{a} |
| Trimedoxime | nd^{c} | nd^{c} | 0.50 ± 0.02^{c} |
| HI-6 | 0.44 ± 0.15 | 50 ± 26 | 9 |
| 6 | nd^{c} | nd^{c} | 0.65^{c} |
| 8 | 0.0094 ± 0.0004 | 15 ± 2 | 0.6 |
| 9 | 0.032 ± 0.001 | 30 ± 3 | 1.1 |
| 11 | 0.56 ± 0.12 | 41 ± 11 | 13 |
| 12 | 0.72 ± 0.07 | 31 ± 6 | 22 |
| 15 | 0.29 ± 0.02 | 21 ± 5 | 14 |

^{a}from the ref. ^{[33]}
^{b}kᵣ₂ = kᵣ/K_{D}
^{c}if [reactivator] [symbol] K_{D}, then there is a linear dependence between k_{obs} and [reactivator]: k_{obs} = (kr/K_{D})[reactivator]. In this case, kᵣ and K_{D} cannot be determined, but kᵣ₂ = kᵣ/K_{D} is the slope of the line.
^{d}Not determined if k_{obs} is < 0.01 min⁻¹ at practical concentration.

TABLE 2
In vitro reactivation of tabun-hAChE

| | kᵣ | K_{D} | kᵣ₂ |
|---|---|---|---|
| Reactivator | (min⁻¹) | (μM) | (mM⁻¹min⁻¹)^{b} |
| Obidoxime | 0.04 ± 0.006 | 250 ± 110 | 0.16 |
| HLö-7 | 0.020 ± 0.0007 | 106 ± 15 | 0.2 |
| Trimedoxime | 0.085 ± 0.005 | 145 ± 25 | 0.7 |
| 11 | — | — | 0.06 ± 0.007 |
| 12 | 0.021 ± 0.001 | 7.1 ± 1.5 | 3 |

In contrast, 4-substituted tetrahydroacridine α-hydroxypyridine amidoximes 6, 8 and 9 bearing C5 alkyl and N-Me containing linkers, respectively, due to the improved affinity exhibited higher reactivation efficiencies (kᵣ₂) for VX-AChE compared to 2-PAM and trimedoxime, but they were less efficient than obidoxime, HLö-7, and HI-6. Yet, it is important to notice that these compounds are the first described efficient amidoxime-based reactivators for VX-inhibited AChE. Remarkably, aldoximes 11, 12 and 15 substituted in position 6 of the pyridine ring by the C5 and C4 alkyl linker were more efficient in reactivating VX-hAChE than 2-PAM, obidoxime, trimedoxime, and HI-6. Although oximes 11, 12 and 15 do not overpass the reactivating efficiency of HLö-7, they display another interesting starting point for further improvement, since they do not bear neither a permanent charge nor a tertiary amine which could hamper the BBB crossing ability.

It is important to point out that oxime 12 reactivates effectively tabun-hAChE (Table 2), which is known to be reluctant to reactivation, due to weak electrophilicity and steric hindrance of the tabun-hAChE adduct. Compound 12 exhibited much higher in vitro reactivation potency compared to all current quaternary pyridinium aldoximes. Despite a lower reactivation rate constant (kᵣ) compared to obidoxime and trimedoxime, 12 due to the significantly improved affinity toward inhibited enzyme (K_{D}) was 4-fold more efficient than trimedoxime, the best pyridinium aldoxime reactivator of tabun-hAChE. The better reactivity of oxime 12 on VX- and tabun-hAChE compared to II suggests that the shorter linker allows better binding to the inhibited enzyme as well as more accurate orientation of the oxime function for displacement of the phosphyl or phosphoramidyl moiety.

Most organophosphate pesticide poisoning results from the formation of a diethylphosphoryl conjugate of hAChE. Testing molecules for their ability to reactivate ethylparaoxon-inhibited hAChE, which results in the formation of the diethylphosphoryl conjugate, provides clues about their general efficacy at countering pesticide poisoning. It appears that compounds 11 and 12 are more efficient than obidoxime for the reactivation of paraoxon-hAChE, and superior to HLö-7 (Table 3). Much like the trend observed in the case of VX, decreasing the linker length from 5 to 4 carbons leads to a 3-fold increase in K_{D} balanced by a 5-fold increase in k.

TABLE 3
In vitro reactivation of paraoxon-hAChE

| | kᵣ | K_{D} | kᵣ₂ |
|---|---|---|---|
| Reactivator | (min⁻¹) | (μM) | (mM⁻¹min⁻¹) ^{b} |
| Obidoxime | 0.81 ^{a} | 32.2 ^{a} | 20 ^{a} |
| HLö-7 | 0.63 ± 0.04 | 210 ± 31 | 3 |
| Trimedoxime | 0.34 ± 0.02 ^{a} | 47.8 ± 6.9 ^{a} | 17 ^{a} |
| 11 | 0.0228 ± 0.0006 | 1.1 ± 0.1 | 20.9 |
| 12 | 0.111 ± 0.002 | 3.6 ± 0.2 | 31 |
| 15 | 0.11 ± 0.01 | 5.7 ± 1.7 | 19 |

^{a} from the ref.^{[34]}
^{a} kᵣ₂ = kr/K_{D}.

### Example 4

### Inhibition of hAChE with Compounds of the Invention

Compounds 7, 10, 11 and 12 were found to inhibit native hAChE with IC₅₀ similar to tacrine and tacrine-pyridyl heterodimer 51 (Table 4).

TABLE 4
Inhibition of hAChE

| compound | IC₅₀ (μM) |
|---|---|
| Tacrine | 0.205 ± 18 |
| | (human erythrocyte |
| | AChE)^{a} |
| 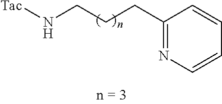 51 | 0.15 ± 0.01 (hAChE) |
| 7 | 0.26 ± 0.01 |
| | (hAChE) |
| 10 | 0.077 ± 0.003 |
| | (hAChE) |
| 11 | 0.26 ± 0.01 |
| | (hAChE) |
| 12 | 1.40 ± 0.03 |
| | (hAChE) |
| 13 | 0.163 |
| | (hAChE) |
| 15 | 2.33 ± 0.18 |
| | (hAChE) |

Interestingly, 4-substituted pyridine aldoxime 10, contrary to 6-substituted compound 11 showed no reactivation of VX-, tabun- and paraoxon inhibited hAChE and a higher inhibition compared to tacrine. This suggests that 4-substitution of pyridine ring favors the binding of tacrine moiety to the catalytic site of the enzyme by contrast to the peripheral site.

1-15. (canceled)

16. Compound of formula (I) 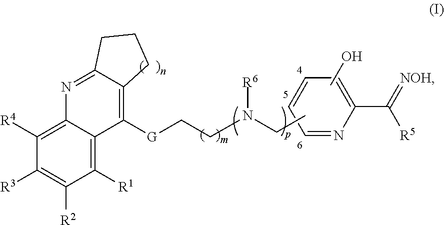
wherein
G is selected from the group consisting of CH₂, O, S, NH, NR, C(O)—NH, C(O)—NR, NH—C(O), NR—C(O), NH—C(S), NR—C(S), NH—NR, NR—NR′, NH—O, NR—O, NH—C—(O)—NH, NR—C(O)—NH, NR—C(O)—NR′, NH—C(S)—NH, NR—C(S)—NH, and NR—C(S)—NR′,
wherein each R or R′ is independently selected from the group consisting of a hydrogen atom, an alkyl group, an acyl group, an aryl group and a heteroaryl group;
n is 1, 2, 3 or 4,
m is 0, 1, 2, 3, or 4,
p is 0 or 1,
R¹, R², R³ and R⁴ are independently selected from the group consisting of a hydrogen atom, a halogen atom, an alkyl group, an alkoxy group, an amine NHR group, an amide NR″—C(O)—R′″ group, an oligopeptide, or a polyethyleneglycol chain, wherein R″ and R′″ have the same definition as R and R′,
R⁵ is a hydrogen atom, a trifluoromethyl group, or a NH₂ group, and
R⁶ is a hydrogen atom, an alkyl group, an aryl group or an acyl group.

17. Compound according to claim 16, wherein m is 2 or 3, preferably m is 3.

18. Compound according to claim 16, wherein n is 2 or 3, preferably n is 2.

19. Compound according to claim 17, wherein n is 2 or 3, preferably n is 2.

20. Compound according to claim 16, wherein p is 0.

21. Compound according to claim 16, wherein the pyridine is substituted in position 6.

22. Compound according to claim 16, wherein R⁵ is a hydrogen atom.

23. Compound according to claim 16, wherein three among R¹, R², R³ and R⁴ are hydrogen atoms.

24. Compound according to claim 23, wherein R¹, R², R³ and R⁴ are hydrogen atoms.

25. Compound according to claim 16, wherein G is NH or S, preferably G is NH.

26. Compound according to claim 16, wherein R⁶ is an alkyl group, preferably a methyl group.

27. Compound according to claim 16, wherein the compound is selected from the group consisting of:
N′,3-dihydroxy-4-{5-[(1,2,3,4-tetrahydroacridin-9-yl)amino]pentyl}pyridine-2-carboximidamide (6)
N′,3-dihydroxy-6-{5-[(1,2,3,4-tetrahydroacridin-9-yl)amino]pentyl}pyridine-2-carboximidamide (7)
(Z)—N′,3-dihydroxy-4-{[methyl({2-[(1,2,3,4-tetrahydroacridin-9-yl)amino]ethyl}) amino]methyl}pyridine-2-carboximidamide (8)
(Z)—N′,3-dihydroxy-4-{[methyl({3-[(1,2,3,4-tetrahydroacridin-9-yl)amino]propyl}) amino]methyl}pyridine-2-carboximidamide (9),
2-[(hydroxyimino)methyl]-4-{5-[(1,2,3,4-tetrahydroacridin-9-yl)amino]pentyl}pyridin-3-ol (10),
2-[(1E)-(hydroxyimino)methyl]-6-{5-[(1,2,3,4-tetrahydroacridin-9-yl)amino]pentyl}pyridin-3-ol (11)
2-[1-(hydroxyimino)methyl]-6-{4-[(1,2,3,4-tetrahydroacridin-9-yl)amino]butyl}pyridin-3-ol (12),
2-[1-(hydroxyimino)methyl]-5-{5-[(1,2,3,4-tetrahydroacridin-9-yl)amino]pentyl}pyridin-3-ol (13),
2-[1-(hydroxyimino)methyl]-6-[5-(1,2,3,4-tetrahydroacridin-9-ylsulfanyl)pentyl]pyridin-3-ol (14),
6-(4-((7-Chloro-1,2,3,4-tetrahydroacridin-9-yl)amino)butyl)-3-hydroxy-picolin-aldehyde oxime (15),
3-Hydroxy-6-(3-((1,2,3,4-tetrahydroacridin-9-yl)amino)propyl) picolinaldehyde oxime (16),
3-Hydroxy-6-(3-((7,8,9,10-tetrahydro-6H-cyclohepta[b]quinolin-11-yl)amino) propyl)picolinaldehyde oxime (17), and
3-Hydroxy-6-(4-((7,8,9,10-tetrahydro-6H-cyclohepta[b]quinolin-11-yl)amino) butyl)picolinaldehyde oxime (18).

28. Pharmaceutical composition comprising at least one compound of formula (I) as defined in claim 16 and at least one pharmaceutically acceptable support.

29. Compound according to claim 16, as an in vitro reactivator of human acetylcholinesterase, wherein the human acetylcholinesterase was inhibited by at least one organophosphorus nerve agents.

30. A method for treating a nervous and/or respiratory failure due to intoxication with at least one organophosphorus nerve agent, comprising administering at least one compound according to claim 16.
